(19) United States
(12) Patent Application Publication
(10) Pub. No.: US 2025/0368933 A1
(43) Pub. Date: Dec. 4, 2025

(54) DEVICES AND METHODS FOR STATIC AND DYNAMIC TISSUE MODULATION

(71) Applicant: Curi Bio, Inc., Seattle, WA (US)

(72) Inventors: Kevin Gray, Seattle, WA (US); Shawn McGuire, Seattle, WA (US); Arjun Acharya, Seattle, WA (US); Nicholas A. Geisse, Lake Forest Park, WA (US); Jacob Fleming, Seattle, WA (US); Cameron Gelber, Los Angeles, CA (US); Christos Michas, Seattle, WA (US)

(73) Assignee: Curi Bio, Inc., Seattle, WA (US)

(21) Appl. No.: 19/108,101

(22) PCT Filed: Sep. 15, 2023
(86) PCT No.: PCT/US2023/074334

§ 371 Date: Feb. 28, 2025

Related U.S. Application Data
(60) Provisional application No. 63/407,300, filed on Sep. 16, 2022.
(60) Provisional application No. 63/507,338, filed on Jun. 9, 2023.

(51) Int. Cl. C12M 3/00; C12M 1/32; C12M 1/42; C12N 5/00

(52) U.S. Cl. CPC C12M 21/08; C12M 23/12; C12M 35/02; C12M 35/04; C12N 5/0062; C12N 2527/00

(57) ABSTRACT

Tissue modulating systems, devices, and methods modulate and/or stimulate numerous tissue specimens, independently and in parallel, within a multi-well plate in such a way to allow simultaneous measurement of functional properties of the tissue specimens. Tissue modulating system includes a first plurality of tissue fixtures, with at least one of the first tissue fixtures extending into each well. The system also includes a tissue fixture modulating apparatus including a second plurality of tissue fixtures, with at least one of the second tissue fixtures extending into each well. The tissue fixture modulating apparatus displaces the second tissue fixture in each well.

### CROSS REFERENCE TO RELATED APPLICATIONS

This application claims the benefit of U.S. Provisional Patent Application No. 63/407,300, filed Sep. 16, 2022, and U.S. Provisional Patent Application No. 63/507,338, filed Jun. 9, 2023, the entire disclosures of which are hereby incorporated by reference in their entireties.

### TECHNICAL FIELD

This disclosure relates generally to the field of tissue research.

### BACKGROUND

Biological muscle tissues undergo mechanical strains in vivo. A substantial body of literature has shown that these strains play a key role in the development of physiological tissues. For instance, within the heart, myocardial tissue experiences mechanical strains in response to blood influx and changes its behavior in response to varying amounts of strains. In another example, skeletal muscle tissues need dynamic mechanical conditioning to properly develop to their full functional capacity but may atrophy if a muscle is left unstressed for a prolonged period.

Similarly, mechanical conditioning of “synthetic” or “engineered” tissues, defined and interchangeably referred to herein as tissues, tissue specimens, or tissue constructs composed of artificially assembled cells and matrices, can be beneficial in mimicking the development and functional behavior of their naturally derived counterparts. Indeed, an apparatus for mechanically stimulating or conditioning such engineered tissues, which is generally designed to control stretching or elongation of the tissues from their native or unstressed length or form as directed by the control protocols set by a user, provides researchers an effective means for simulating physiologically relevant mechanical strains in vitro.

Devices have been developed to apply stimulation or stresses to the engineered tissues. See, e.g., U.S. Patent Publication No. US 2019/0029549 and U.S. Pat. No. 6,048,723. However, some such devices cannot mechanically stretch engineered tissues. Other such devices allow for mechanical stimulation of multiple tissues simultaneously; however, in operation, each tissue in these devices is exposed to roughly the same type, form, or strength of stimulus because these devices are designed to apply strain to the entire tissue-culture device containing multiple tissues, instead of applying an individualized strain to each tissue independent of other tissues under test. As a result, researchers cannot effectively test or condition multiple stimulation regimes with a single device. Furthermore, if certain tissue specimens within a tissue-culture device develop differently due to genetic disorders or other physiological variability, the stimuli applied to each such tissue specimen cannot be easily tuned to interplay with each tissue specimen's physiological state or characteristics and evoke optimal physiological behavior of such tissue specimens under test.

Thus, a tissue modulating system that can precisely apply mechanical strains to one or more tissue specimens and optionally independently apply individualized strain profiles to multiple tissue specimens, e.g., in parallel within a standard multi-well form of tissue-culture plates, will be of significant utility. Additionally, the ability to modulate individual tissue inputs (e.g., strain and stimulation current) based on feedback from real-time measurement of functional outputs (e.g., contraction force) for one or more tissue specimens overcomes shortcomings of other devices.

### SUMMARY

The present disclosure provides tissue modulating systems, devices, and methods capable of modulating and/or stimulating numerous tissue specimens, independently and in parallel, within a multi-well tissue culture plate in such a way to allow simultaneous measurement of functional properties of the tissue specimens.

In an aspect, the present disclosure provides a tissue modulating system configured for use with a multi-well plate comprising a plurality of wells. The system may or may not comprise the multi-well plate. The tissue modulating system includes a first plurality of tissue fixtures, wherein at least one tissue fixture of the first plurality of tissue fixtures may be configured to extend into each well of the plurality of wells. The system also includes a tissue fixture modulating apparatus comprising a second plurality of tissue fixtures, wherein at least one tissue fixture of the second plurality of tissue fixtures extends into each well of the plurality of wells, wherein the tissue fixture modulating apparatus may be configured to displace, within each well, the at least one tissue fixture of the second plurality of tissue fixtures.

In any embodiment, each tissue fixture of the second plurality of tissue fixtures may have a greater stiffness than each tissue fixture of the first plurality of tissue fixtures.

In any embodiment, the first plurality of tissue fixtures may be a plurality of flexible posts, hooks, and/or clamps, and the second plurality of tissue fixtures may be a plurality of rigid posts, hooks, and/or clamps.

In any embodiment, each tissue fixture of the first plurality of tissue fixtures may have a greater stiffness than each tissue fixture of the second plurality of tissue fixtures.

In any embodiment, the first plurality of tissue fixtures may be a plurality of rigid posts, hooks, and/or clamps, and the second plurality of tissue fixtures may be a plurality of flexible posts, hooks, and/or clamps.

In any embodiment, the system (e.g., the tissue fixture modulating apparatus) may further include a well plate lid securable to the multi-well plate, wherein at least one of the first plurality of tissue fixtures or the second plurality of tissue fixtures attaches to the well plate lid.

In any embodiment, at least one of the first plurality of tissue fixtures and/or the second plurality of tissue fixtures may attach to the well plate lid and/or extend through the well plate lid.

In any embodiment, the second plurality of tissue fixtures may pivot about a pivot point.

In any embodiment, the second plurality of tissue fixtures may extend away from a beam coupled with a pivot point.

In any embodiment, the tissue fixture modulating apparatus may include at least one actuator configured to pivot or translate the beam.

In any embodiment, the actuator may contact a lever extending away from a beam.

In any embodiment, the system (e.g., the tissue fixture modulating apparatus) may include a plurality of tissue fixture limiters configured to extend into the plurality of wells.

In any embodiment, a distal end of each tissue fixture limiter may be configured to selectively intercept movement of at least one of the second plurality of tissue fixtures.

In any embodiment, the plurality of tissue fixture limiters may be independently movable between a fully retracted position and a fully deployed position.

In any embodiment, the system may further include a fixture bridge, optionally extending across the plurality of wells, the fixture bridge including the plurality of tissue fixture limiters.

In any embodiment, the system may further include a plurality of electrodes configured for extending into the plurality of wells.

In any embodiment, the system (e.g., the tissue modulating apparatus) may include one or more actuators configured to displace one or more tissue fixtures of the second plurality of tissue fixtures. One or more of the actuators may be manual, electronic, pneumatic, or magnetic.

In any embodiment, the system (e.g., the tissue modulating apparatus) may include a plurality of actuators, each actuator of the plurality of actuators being configured to displace a different tissue fixture of the second plurality of tissue fixtures (e.g., independent actuation of each tissue fixture). One or more of the actuators may be manual, electronic, pneumatic, or magnetic.

In any embodiment, the system may include a non-transitory computer-readable storage medium storing instructions that when executed by a processor cause the processor to actuate each actuator, optionally independently from each other actuator of the plurality of actuators.

In any embodiment, the system may include a non-transitory computer-readable storage medium storing instructions that when executed by a processor cause the processor to actuate the tissue fixture modulating apparatus.

In any embodiment, the system may include a plurality of electrodes configured to extend into the plurality of wells, wherein the instructions further include applying a voltage to the plurality of electrodes, optionally while actuating the tissue fixture modulating apparatus. In any embodiment described herein, the instructions may cause the system to perform different tissue modulating protocols (including any one or more of the tissue modulating protocols described herein) contemporaneously in different wells, e.g., through independent control of the tissue fixture modulating apparatus and/or stimulation electrodes for different wells.

In any embodiment, the system may include (or not include) the multi-well plate.

In another aspect, the present disclosure provides methods for modulating a tissue specimen, performing a tissue modulating protocol, and for using tissue modulating systems and tissue fixture modulating apparatuses.

In another aspect, the present disclosure provides methods for modulating a tissue specimen. The methods include attaching a tissue specimen between first tissue fixture and a second tissue fixture; stretching the tissue specimen while applying a stimulation current to the tissue specimen during a fused tetanus state or a fatigue state of the tissue specimen; allowing the first tissue fixture to move toward the second tissue fixture while applying the stimulation current after stretching the tissue specimen; sensing a position of at least one of the first tissue fixture or the second tissue fixture; and determining a tissue characteristic of the tissue specimen based on the position.

In another aspect, the present disclosure provides methods for modulating a tissue specimen. The methods include attaching a tissue specimen between a first tissue fixture and a second tissue fixture; applying a stimulation current to the tissue specimen; stretching the tissue specimen during a relaxation state when the stimulation current may be not applied to the tissue specimen; allowing the first tissue fixture to move toward the second tissue fixture after stretching the tissue specimen; sensing a position of at least one of the first tissue fixture or the second tissue fixture; and determining a tissue characteristic of the tissue specimen based on the position.

In another aspect, the present disclosure provides methods for modulating a tissue specimen. The methods include attaching a tissue specimen between a first tissue fixture and a second tissue fixture; moving a first tissue fixture limiter into a partially or fully deployed position obstructing a travel path of the first tissue fixture in a first direction; causing the tissue specimen to exert a contraction force such that the first tissue fixture contacts the first tissue fixture limiter; moving the first tissue fixture limiter into a partially or fully retracted position while the tissue specimen exerts the contraction force; moving a second tissue fixture limiter into a partially or fully deployed position obstructing the travel path of the first tissue fixture in a second direction; and moving the second tissue fixture limiter into a partially or fully retracted position.

In another aspect, the present disclosure provides methods for modulating a tissue specimen. The methods include attaching a tissue specimen between a first tissue fixture and a second tissue fixture; moving a first tissue fixture limiter into a partially or fully deployed position obstructing a travel path of the first tissue fixture in a first direction; causing the tissue specimen to exert a contraction force such that the first tissue fixture contacts the first tissue fixture limiter; reducing an effective stiffness of the first tissue fixture by moving the first tissue fixture limiter; moving a second tissue fixture limiter into a partially or fully deployed position obstructing the travel path of the first tissue fixture in a second direction; and moving the second tissue fixture limiter into a partially or fully retracted position.

In another aspect, the present disclosure provides methods for modulating a tissue specimen. The methods include attaching a tissue specimen between a first tissue fixture and a second tissue fixture; allowing the tissue specimen to reach a first resting state; determining a first tissue characteristic of the tissue specimen in the first resting state; moving the first tissue fixture toward the second tissue fixture when the tissue specimen may be in the first resting state; allowing the tissue specimen to reach a second resting state; and determining a second tissue characteristic of the tissue specimen in the second resting state.

In another aspect, the present disclosure provides methods for modulating a tissue specimen. The methods include attaching a tissue specimen between a first tissue fixture and a second tissue fixture; separating the first tissue fixture from the second tissue fixture, thereby modulating the tissue specimen from a first length to a second length; holding the tissue specimen at the second length by modulating a tissue stretch and an electrical stimulation; allowing the tissue specimen to return to the first length; and holding the tissue specimen in the first length by modulating the tissue stretch while the tissue specimen relaxes.

In another aspect, the present disclosure provides methods for modulating a tissue specimen. The methods include attaching a tissue specimen between a first tissue fixture and a second tissue fixture; moving a first tissue fixture limiter into a partially or fully deployed position obstructing a travel path of the first tissue fixture in a first direction; and causing the tissue specimen to exert a contraction force such that the first tissue fixture contacts the first tissue fixture limiter.

In any of the foregoing methods, the method may include attaching a tissue specimen between a first tissue fixture and a second tissue fixture in a well of a multi-well plate, and performing a tissue modulating protocol on the tissue specimen, wherein the tissue modulating protocol includes stretching the tissue specimen by actuating at least one of the first tissue fixture or the second tissue fixture.

In any of the foregoing methods, the first tissue fixture may be a rigid or flexible tissue fixture (e.g., a rigid post) and the second tissue fixture may be a flexible or rigid tissue fixture (e.g., a flexible post).

In any of the foregoing methods, the tissue modulating protocol may include stretching the tissue specimen while applying a stimulation current to the tissue specimen.

In any of the foregoing methods, the method (e.g., the tissue modulating protocol) may include applying the stimulation current to the tissue specimen, optionally causing the tissue specimen to achieve a fused tetanus state when stretched.

In any of the foregoing methods, the stimulation current may cause the tissue specimen to recruit fewer than all sarcomeres of the tissue specimen in the fused tetanus state when stretched.

In any of the foregoing methods, stretching the tissue specimen may be performed by the separating the first tissue fixture from the second tissue fixture.

In any of the foregoing methods, the method may include separating the first tissue fixture from the second tissue fixture, e.g., by displacing a rigid tissue fixture (e.g., rigid post).

In any of the foregoing methods, the tissue modulating protocol may further include allowing the first tissue fixture to move toward the second tissue fixture while applying the stimulation current after separating the first tissue fixture from the second tissue fixture.

In any of the foregoing methods, applying the stimulation current to the tissue specimen may cause the tissue specimen to achieve a fused tetanus state, wherein the tissue specimen may be in a fatigue state when stretched.

In any of the foregoing methods, the method (e.g., the tissue modulating protocol) may further include allowing the first tissue fixture to move toward the second tissue fixture while applying the stimulation current after stretching the tissue specimen.

In any of the foregoing methods, stretching the tissue specimen may include repetitively separating the first tissue fixture from the second tissue fixture, e.g., when no stimulation is applied to the tissue specimen.

In any of the foregoing methods, the method (e.g., the tissue modulating protocol) may include applying a stimulation current to the tissue specimen, e.g., causing the tissue specimen to achieve a fused tetanus state; and stretching the tissue specimen during a relaxation state when the stimulation current may be not applied to the tissue specimen.

In any of the foregoing methods, stretching the tissue specimen may be performed by separating the first tissue fixture from the second tissue fixture.

In any of the foregoing methods, separating the first tissue fixture from the second tissue fixture may be performed by displacing a rigid post.

In any of the foregoing methods, the method may further include measuring a deflection of the second tissue fixture during and/or after stretching the tissue specimen.

Any combination of the foregoing features and steps may be combined in additional embodiments of the present disclosure.

### BRIEF DESCRIPTION OF THE DRAWINGS

To easily identify the discussion of any particular element or act, the most significant digit or digits in a reference number refer to the figure number in which that element is first introduced.

FIG. 1A illustrates a perspective view of a tissue modulating system according to a representative embodiment of the present disclosure.

FIG. 1B illustrates an exploded perspective view of a tissue modulating device of the tissue modulating system of FIG. 1A.

FIG. 1C illustrates another exploded view of the tissue modulating device of the tissue modulating system of FIG. 1A.

FIG. 1D illustrates a section view of tissue modulating device of the tissue modulating system of FIG. 1A.

FIG. 1E illustrates a perspective view of a first aspect of a tissue modulating apparatus of the tissue modulating device of FIG. 1B-FIG. 1D.

FIG. 1F illustrates a perspective view of a second aspect of a tissue modulating apparatus of the tissue modulating device of FIG. 1B-FIG. 1D.

FIG. 1G schematically illustrates additional aspects of the tissue modulating system of FIG. 1A.

FIG. 2A-FIG. 2B illustrates aspects of another tissue modulating device of the present disclosure.

FIG. 3 illustrates a representative tissue fixture limiter of the present disclosure.

FIG. 4 illustrates a pressure-volume relationship of a heart.

FIG. 5 illustrates a method of modulating a tissue specimen according to the present disclosure.

FIG. 6 illustrates another method of modulating a tissue specimen according to the present disclosure.

FIG. 7A illustrates an exploded perspective view of aspects of another representative tissue modulating device of the present disclosure.

FIG. 7B illustrates an exploded view of aspects of a tissue fixture modulating apparatus of the tissue modulating device of FIG. 7A.

FIG. 7C illustrates a section view of aspects of the tissue modulating device of FIG. 7A.

FIG. 7D-FIG. 7E schematically illustrate aspects of the tissue modulating device of FIG. 7A in a first state and a second state, respectively.

FIG. 8 illustrates a section view of aspects of a tissue fixture modulating apparatus with a stimulation electrode according to the present disclosure.

FIG. 9 illustrates an exploded perspective view of aspects of another representative tissue modulating device of the present disclosure.

FIG. 10 illustrates a method for modulating a tissue specimen according to the present disclosure.

FIG. 11 illustrates another method for modulating a tissue specimen according to the present disclosure.

FIG. 12 illustrates an eccentric strain tissue modulating protocol according to the present disclosure.

FIG. 13 illustrates an eccentric stretch during fatigue tissue modulating protocol according to the present disclosure.

FIG. 14 illustrates an eccentric stretch with partial recruitment tissue modulating protocol according to the present disclosure.

FIG. 15 illustrates another method for modulating a tissue specimen according to the present disclosure.

FIG. 16 illustrates a high strain during relaxation tissue modulating protocol according to the present disclosure.

FIG. 17 illustrates a repetitive high rate strain no stimulation tissue modulating protocol according to the present disclosure.

FIG. 18 illustrates a stiffness tissue modulating protocol according to the present disclosure.

FIG. 19 illustrates a force-length relationship tissue modulating protocol according to the present disclosure.

FIG. 20 illustrates still another method for modulating a tissue specimen according to the present disclosure.

FIG. 21 illustrates still another method for modulating a tissue specimen according to the present disclosure.

The foregoing aspects and many of the attendant advantages of the claimed subject matter will become more readily appreciated by reference to the following detailed description, when taken in conjunction with the accompanying drawings.

### DETAILED DESCRIPTION

This disclosure provides tissue modulating systems and devices, in addition to methods for modulating tissue specimens through stretching and/or compression, optionally with electrical stimulation, as well as for determining various tissue characteristics of such tissue specimens, e.g., contractile force, stiffness, elasticity modulus, beat frequency, tissue displacement waveform properties, biomarker emissions, etc. For example, the present disclosure provides tissue modulating systems and tissue fixture modulating apparatuses which stretch, contract, and/or stimulate tissue specimens. Tissue specimens may include, for example, any natural or engineered (synthetic) musculoskeletal tissues including skeletal muscle, smooth muscle, cardiac muscle, tendons, and ligaments. Tissue specimens may also include non-musculoskeletal tissue types, including pulmonary, tracheal, intestinal, hepatic and neuromuscular, and tumors. Representative and non-limiting tissue specimens contemplated by the present disclosure have a diameter of about 0.3 mm to about 2.0 mm (e.g., about 1.0 mm) and a length of about 1.0 mm to about 15.0 mm, for example about 5.0 mm to about 10.0 mm (e.g., about 7.0 mm).

The tissue modulating systems may include one or more tissue modulating devices, which may include any one or more of: a tissue fixture modulating apparatus, tissue fixtures, tissue fixture limiter, multi-well plate, well plate lid, stimulation plate, a sensor array, computing elements such as a processor and a computer-readable storage medium storage instructions for execution of one or more tissue modulating protocols, user interfaces, and other elements.

Further still, the present disclosure provides methods for determining a tissue characteristic and tissue modulating protocols which may be performed on tissue specimens with or without the tissue modulating systems described herein. Representative embodiments will now be described.

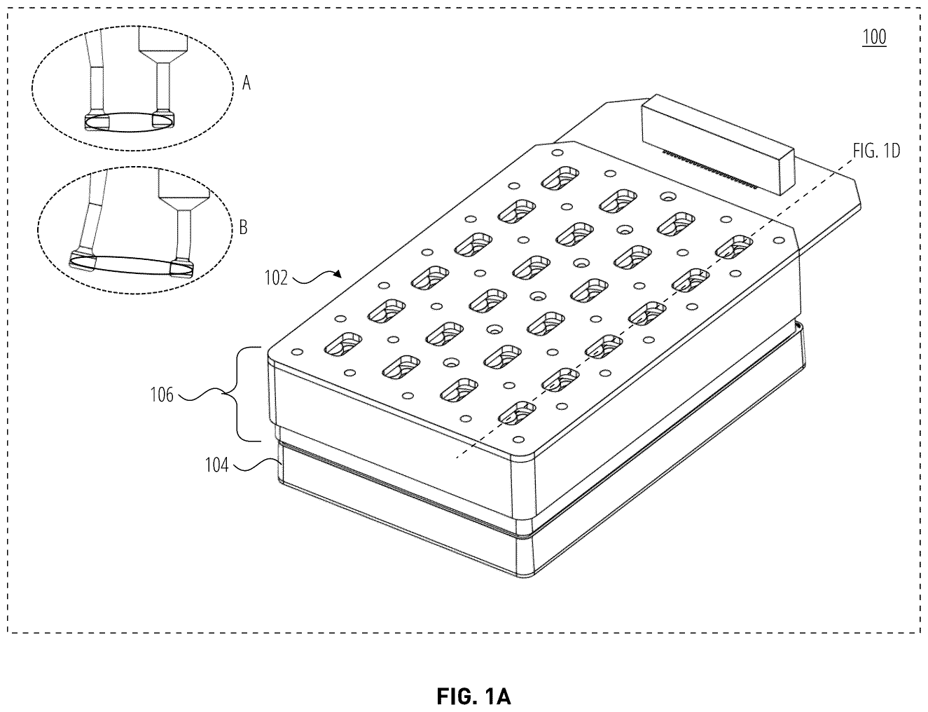

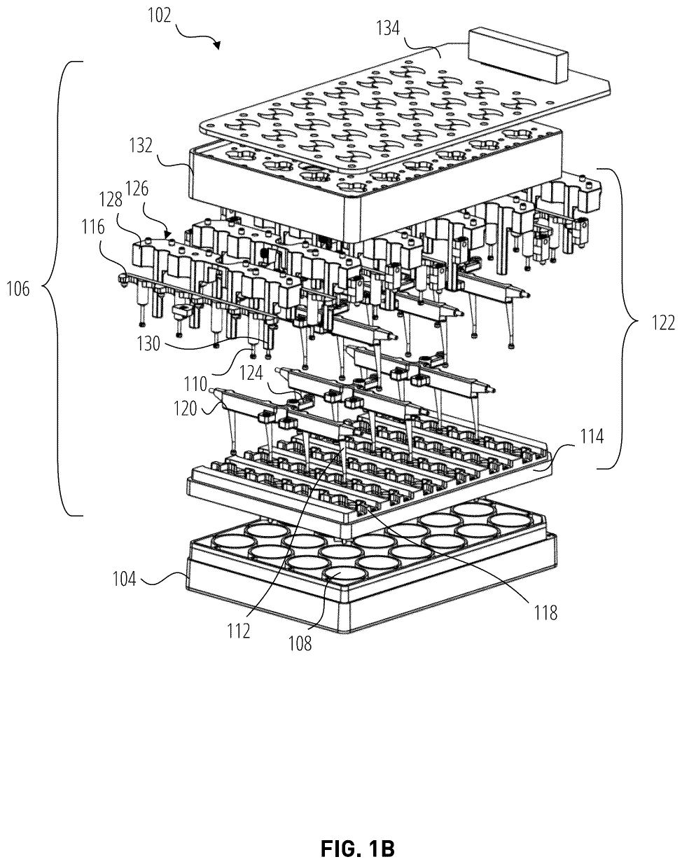

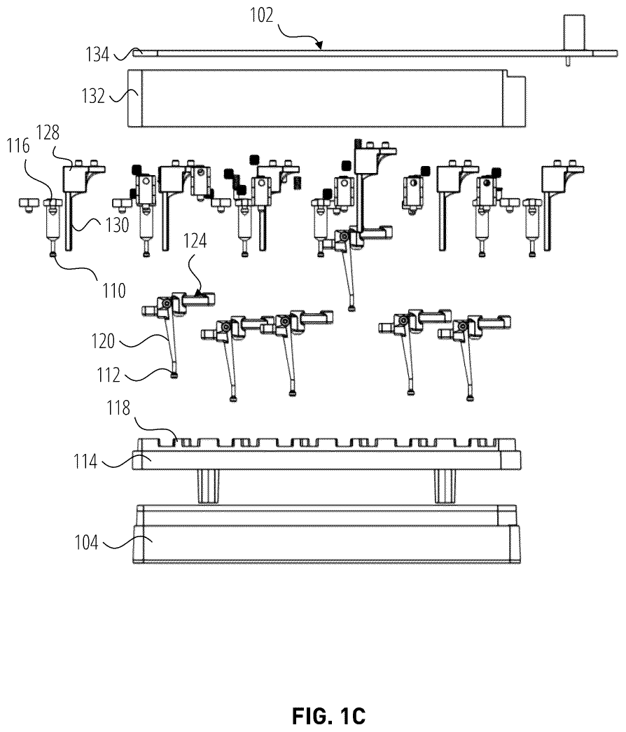

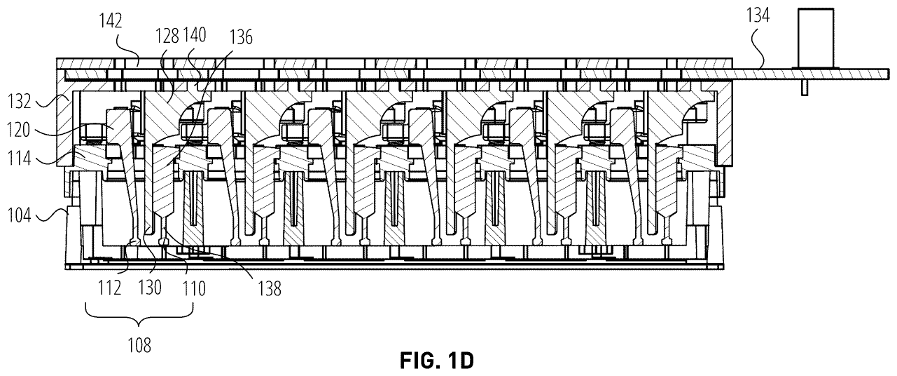

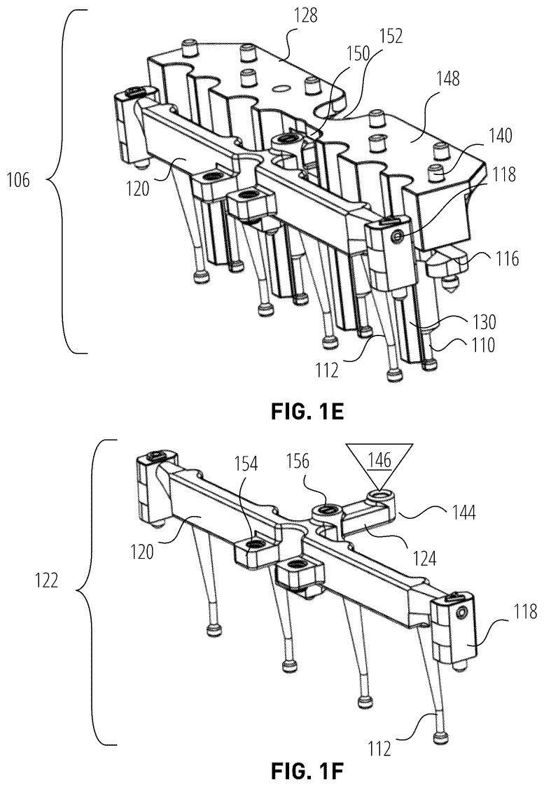

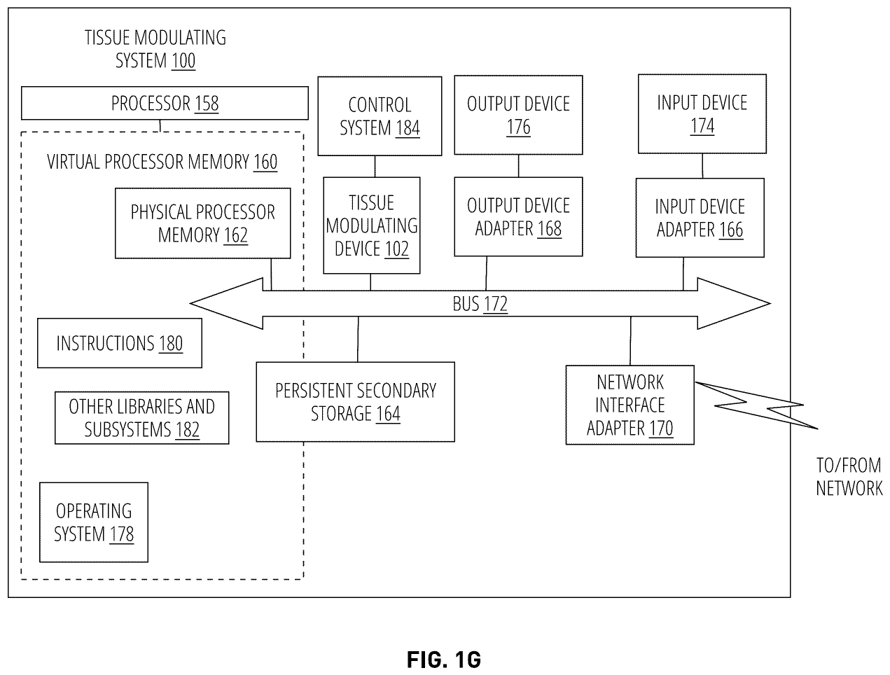

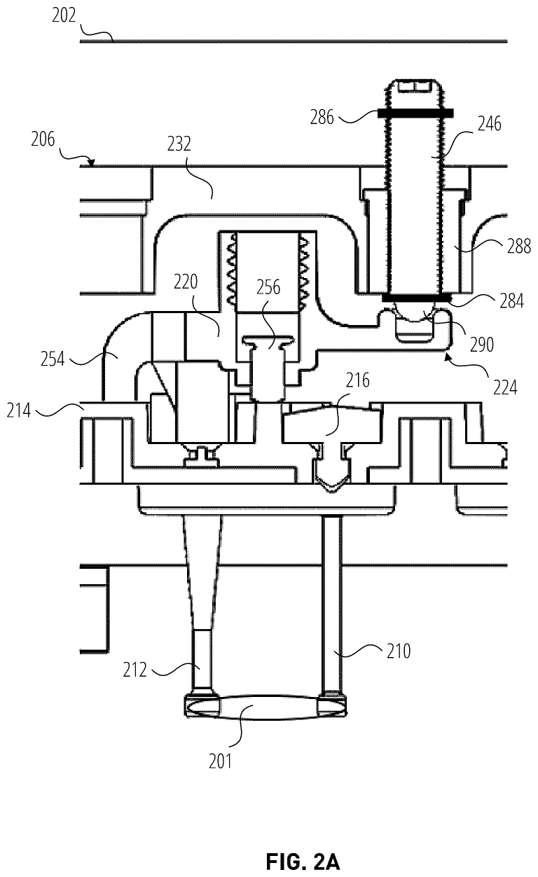

FIG. 1A-FIG. 1F show aspects of one representative tissue modulating system 100 which includes a representative tissue modulating device 102 configured to modulate at least one tissue specimen between an unstretched state (see insert A) and one or more stretched and/or compressed states (see insert B). The following description of FIG. 1A-FIG. 1C provides a general introduction to the tissue modulating device 102. FIG. 1D-FIG. 1F provide additional details of subsystems thereof.

As described below, the tissue modulating device 102 enables parallel modulation and analysis of numerous tissue specimens, e.g., 24, 96, or more tissue specimens in a single device. In particular, the tissue modulating device 102 enables the contemporaneous execution of one or more tissue modulating protocols on the tissue specimens. The tissue modulating protocols cause physiological responses in the tissue specimens, e.g., injury, thereby facilitating high throughput analysis of said tissue specimens.

Tissue modulating device 102 is an electromechanical apparatus which may be utilized individually or in parallel with other such apparatuses to increase throughput. The device 102 is attachable to a multi-well plate 104 and includes a tissue fixture modulating apparatus 106 configured to modulate one or more tissue specimens between the unstretched state (FIG. 1A, inset A) and the stretched state (FIG. 1A, inset B). Some embodiments include the multi-well plate 104; however, the multi-well plate 104 may optionally form a separate consumable element separate from the multi-well plate 104. As described below, the tissue modulating device 102 includes a plurality of tissue fixtures within each well of the multi-well plate 104, each tissue fixture being configured for attachment to a tissue specimen (such as by culturing). For example, a tissue specimen may be attached between two tissue fixtures within each well. The tissue fixtures may be posts, hooks, clamps, or other fixture type capable of attaching to a tissue specimen via tissue growth around such fixture or mechanical engagement. Within each well, two tissue fixtures may have the same or different properties. For example, a relatively rigid tissue fixture and a relatively flexible tissue fixture may extend into each well.

In the non-limiting embodiments described herein, the tissue fixtures generally include posts, e.g., flexible posts and rigid posts. For example, in the tissue modulating device 102, the tissue fixtures include a plurality of rigid posts and a plurality of flexible posts. At least one flexible post and at least one rigid post extends into each well of the multi-well plate such that the respective distal ends are adjacent and spaced apart. A tissue specimen may be cultured in a growth medium within each well such that it attaches between the distal ends of the rigid post and flexible post.

Although the representative embodiments described generally include two tissue fixtures having different properties in a single well (e.g., a rigid post and a flexible post having different stiffness properties in each well), it is not necessary for two tissue fixtures within a single well to have different properties. For example, in some embodiments, at least two identical tissue fixtures (e.g., two rigid posts or two flexible posts) may extend into each well.

The tissue modulating system 100 may optionally include any of the computing elements shown in FIG. 1G. In some embodiments, the tissue modulating device 102 comprises some or all of the computing elements of FIG. 1G, namely a processor and computer-readable storage medium storing instructions thereon which cause the tissue modulating device 102 to execute one or more tissue modulating protocols as described herein. In any embodiment described herein, the instructions may cause the system to perform different tissue modulating protocols (including any one or more of the tissue modulating protocols described herein) contemporaneously in different wells, e.g., through independent control of the tissue fixture modulating apparatus 106 and/or stimulation electrodes for different wells.

Turning to the exploded views of FIG. 1B and FIG. 1C, the multi-well plate 104 is a plate formed of polystyrene, polycarbonate, PET, or similar material and having a well 108 (e.g., 24, 96, 384, 1536, or other number of wells), each being sized and otherwise configured to receive at least one flexible post, at least one rigid post, a tissue specimen adhered between the flexible post and rigid post in a growth medium, and optionally a tissue fixture limiter. The wells 108 each have a closed bottom in the representative embodiment shown; in other embodiments, the wells may have an open bottom, or a transparent bottom to facilitate imaging. Representative multi-well plates 104 include, but are not limited to, NanoSurface plates sold by Curi Bio, Inc. of Seattle, Washington, and Corning® and Falcon® cultureware plates sold by Corning Incorporated of Corning, New York. As stated previously, some embodiments of the tissue modulating device 102 include the multi-well plate 104; however, the multi-well plate 104 may be a separate consumable from the tissue modulating device 102.

A first plurality of tissue fixtures comprises a plurality of flexible posts 110 and second plurality of tissue fixtures comprises a plurality of rigid posts 112. In any embodiment herein, the first and/or second plurality of tissue fixtures may be characterized as a first and/or second plurality of flexible tissue fixtures or rigid tissue fixtures. In the embodiment shown, at least one of the flexible posts 110 and at least one of the rigid posts 112 (e.g., a post pair) extends into each well 108 of the multi-well plate 104.

The term “rigid post”/rigid tissue fixture and “flexible post”/rigid tissue fixture may be defined absolutely and/or relatively. For example, in some embodiments, each rigid tissue fixture (e.g., rigid post 112) has a greater force-to-displacement relationship, e.g., at one point along the length of the post (e.g., a greater stiffness at the distal end) and/or a different Young's modulus than the flexible tissue fixture (e.g., flexible post 110) in the same well. In some embodiments, the foregoing relationship (e.g., between the stiffness of the rigid post and the flexible post) may have a ratio of 1×-1,000×, e.g., about 1× to about 500×, about 5× to about 500×, or about 10× to about 300×. In absolute terms, by way of example, not limitation, in some embodiments, each rigid post 112 has a stiffness of about 1,000 N/m to about 10,000 N/m, for example about 10 N/m to about 30 N/m (e.g., about 12 N/m or 24 N/m). In some embodiments, each flexible post 110 has a stiffness of about 0.1 N/m to about 5 N/m, e.g., about 2 N/m.

Any combination of the foregoing rigid and flexible posts may be utilized. In some embodiments, some or all of the tissue fixtures are part of the tissue fixture modulating apparatus 106. For example, in some embodiments, the rigid posts 112 form part of the tissue fixture modulating apparatus 106 and the actuation assembly (described below); however, the flexible posts 110 are separate therefrom. In other embodiments, the flexible posts 110 and rigid posts 112 are part of the tissue fixture modulating apparatus 106 and the actuation assembly. In still other embodiments, the flexible posts 110 form part of the tissue fixture modulating apparatus 106 and the actuation assembly; however, the rigid posts 112 are separate therefrom. In some embodiments, some or all of the tissue fixtures form part of the well plate lid 114. For example, in some embodiments, the well plate lid 114 is an assembly that includes the flexible posts 110.

A well plate lid 114 couples to an upper surface of the multi-well plate 104, e.g., after each well is provided with cells and growth medium. Well plate lid 114 serves as a support for the flexible posts 110 and the tissue fixture modulating apparatus 106. In the embodiment shown, the well plate lid 114 is a lattice comprising apertures extending therethrough directly over the wells 108. The flexible posts 110 extend away from a post beam 116, which attaches to the well plate lid 114 such that each flexible post 110 extends through an aperture thereof and into one of the wells 108. In any embodiment, the well plate lid 114 is part of the tissue modulating device 102, tissue fixture modulating apparatus 106, and/or an actuation assembly (described below).

Tissue fixture modulating apparatus 106 couples to the well plate lid 114 or the plate 104 and directly acts on at least one tissue fixture within each well such that a tissue specimen attached between two tissue fixtures is stretched, compressed, or otherwise modulated. Tissue fixture modulating apparatuses may directly pivot, translate, rotate, bend, extend, retract, or otherwise directly act on all or a portion of one or more tissue fixtures. In the embodiment shown, the tissue fixture modulating apparatus 106 comprises the well plate lid 114. In other embodiments, the tissue fixture modulating apparatus 106 does not comprise the well plate lid 114.

In some embodiments, the tissue fixture modulating apparatus 106 directly varies a distance between two tissue fixtures within each well (e.g., between a rigid post and a flexible post). For example, in some embodiments, the tissue fixture modulating apparatuses directly displace the flexible post and/or the rigid post of each post pair. In other embodiments, the tissue fixture modulating apparatus 106 stretches or compresses the tissue specimen by rotating at least one tissue fixture (e.g., rotating a rigid post about its longitudinal axis). Advantageously, using a tissue fixture modulating apparatus to stretch or compress a tissue specimen enables the execution of any number of tissue modulating protocols (including but not limited to those expressly described herein), which is useful to determine one or more tissue characteristics such as strain, stress, or Young's Modulus, and to assess the change in tissue characteristics over time.

The tissue fixture modulating apparatus 106 shown includes the rigid posts 112, each of which extends into one of the wells 108 of the multi-well plate 104. In the embodiment shown, each of the rigid posts 112 attaches indirectly to the well plate lid 114 (via structure described below) and extends through an aperture of the well plate lid 114 and into one well 108 of the multi-well plate 104.

The tissue fixture modulating apparatus 106 acts directly on the rigid posts 112 by pivoting said rigid posts 112 about at least one pivot point 118, e.g., a journal or fulcrum disposed on a post extending from an upper surface of the well plate lid 114 or multi-well plate 104. In particular, the rigid posts 112 each extend away from one of numerous pivoting beams 120, each of which is coupled with one of the pivot points 118. Each pivoting beam 120 extends across a plurality of the wells 108.

The tissue fixture modulating apparatus 106 comprises an actuation assembly 122 configured to act on the rigid posts 112, e.g., to cause one or more of the rigid posts 112 to pivot, translate, rotate, bend, extend, retract. In some embodiments, the actuation assembly 122 acts on a plurality of tissue fixtures simultaneously (e.g., global control). In some embodiments, the actuation assembly 122 acts on two or more tissue fixtures independently from each other.

In the embodiment shown, the actuation assembly 122 includes an arm actuator 124 that pivots one or more of the pivoting beams 120, which in turn causes each rigid post 112 extending from that pivoting beam 120 to pivot within the corresponding well 108. The arm actuator 124 comprises an actuator, e.g., a linear actuator with a lead screw, in contact with an actuation lever extending away from the respective pivoting beam 120.

Advantageously, utilizing a common pivoting beam for a plurality of rigid posts enables a single arm actuator 124 to pivot multiple rigid posts 112, as described below. In the embodiment shown, the tissue fixture modulating apparatus 106 includes six pivoting beams 120, each having four rigid posts 112 extending away therefrom (corresponding to the 24-well plate 104). Other tissue fixture modulating apparatuses have greater or fewer pivoting beams, each with fewer, the same, or greater number of rigid posts extending away therefrom based upon the corresponding multi-well plate. For example, a tissue fixture modulating apparatus configured for attachment to a 96-well plate may comprise 8 pivoting beams, each with 12 rigid posts extending away therefrom.

Tissue fixture modulating apparatus 106 comprises an optional tissue fixture limiting system 126 that selectively constrains movement of one or more of the flexible posts 110 and/or rigid posts 112. Advantageously, such tissue fixture limiting system 126 enables stretching or strain of relatively stiff tissue specimens and/or enables isometric tissue modulating protocols, i.e., where the length of the tissue specimen does not change despite contraction. In the embodiment shown, the tissue fixture limiting system 126 includes a fixture bridge 128 including a plurality of tissue fixture limiters 130 formed as rigid rods or posts extending away therefrom such that a distal end of each tissue fixture limiter 130 extends into one of the wells 108.

Generally, the tissue fixture limiters may include a beam, rod, dowel, pin, post, or similar element that can be retractably inserted into or adjacent to the flexible post and which has a stiffness comparable to the rigid post in order to prevent deflection of the flexible post. In some embodiments such as the tissue fixture limiter 130, the tissue fixture limiter is not inserted into the flexible post, but forms a “stop” at a distance away from the flexible post that mechanically prevents at least a portion of the flexible post from deflecting beyond the tissue fixture limiter, thereby increasing its effective stiffness. To clarify, a portion of all of the flexible post may still bend or deflect; however, the tissue fixture limiter obstructs at least a portion of the flexible post from bending beyond the point at which it makes contact with the tissue fixture limiter. The position of the fixture can be controlled in a manual or automated manner, where the fixture is moved in a lateral or vertical manner relative to the flexible post.

Particularly, the distal end of each tissue fixture limiter 130 is positioned adjacent to (nearly abutting) a distal end of one of the flexible post 110. In such a manner, the distal end of each tissue fixture limiter 130 acts as a stop in at least one dimension to the deflection of the distal end of the flexible post 110. In some embodiments, the distal end of each tissue fixture limiter 130 is configured to selectively retract from the distal end of the corresponding flexible post 110.

Tissue fixture modulating apparatus 106 may include additional elements, for example one or more actuator lids 132 (e.g., a stimulation lid) and a printed circuit board 134.

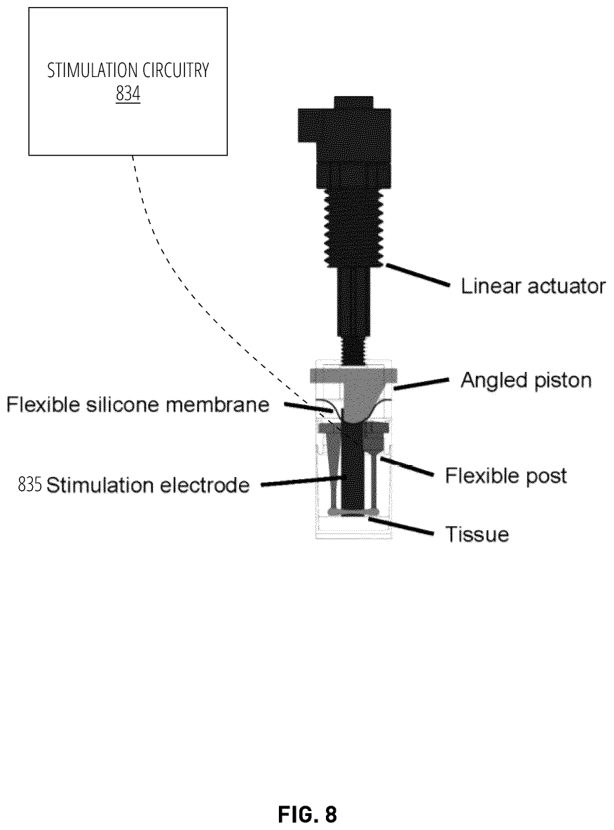

The printed circuit board 134 shown in FIG. 1B is a stimulation lid printed circuit board comprising circuitry for applying a voltage (e.g., a variable or a constant voltage) to electrodes extending into each of the wells 108. To this end, any tissue modulating system 100 and/or tissue modulating device 102 can be further improved with optional integration of electrical stimulation means for applying a stimulation current to the tissue specimens. FIG. 8 shows a representative electrode configuration. Additional representative stimulation structures are described in PCT Publication No. WO2021173887A1, which is hereby incorporated by reference. Such electrodes can apply a stimulation current to the tissue specimen(s), causing the tissue specimens to contract, e.g., as part of a tissue modulating protocol.

The printed circuit board 134 may also include and/or be operably connected to circuitry and sensors equipped for detecting, tracking and computing static and dynamic stretching motions of the tissue specimens under test. For example, the printed circuit board 134 may include or be operably connected to an optional control system 184 (described below) comprising one or more optical sensors (e.g., a camera), one or more magnetometers, or other sensor disposed underneath or above each well of the well 108 to measure in real time the position and/or positional change, at one or more time points, of the tissue specimens under test. Such a sensor array is configured to sense a position and/or a positional change of one or more tissue fixtures. Representative sensors include, for example a mounted optical sensor array, under each well, for measuring distance between ends of rigid and flexible posts. Representative sensors and sensor arrays include those described in International Publication No. WO 2021/173887 A1 and International Publication No. WO 2023/154775 A1 (both to Curi Bio, Inc.), the entireties of which are incorporated by reference.

FIG. 1D shows a first section view of the tissue modulating device 102 of FIG. 1A in order to show the interrelationship between elements within the well 108 and between elements of the tissue fixture modulating apparatus 106 and the tissue fixture limiting system 126. The relationships described with respect to well 108 are true with respect to other wells of the tissue modulating device 102.

As shown, the flexible post 110 has a relatively rigid upper portion 136 and a relatively flexible distal portion 138 extending therefrom. In other embodiments, one or more of the flexible posts and/or rigid posts have a geometrically uniform construction. Distal ends of the flexible post 110 and rigid post 112 each extend into well 108 in a spaced-apart adjacent relationship, e.g., separated by about 3 mm to about 10 mm. In use, a tissue specimen is attached (e.g., by culturing) in between these distal ends.

The tissue fixture limiter 130 extends away from the removable fixture bridge 128, which is secured in this embodiment to the actuator lid 132 via pin 140. The fixture bridge 128 is selectively removable, e.g., by removing the actuator lid 132 and printed circuit board 134 first, then extracting the tissue fixture limiter 130 from the multi-well plate 104.

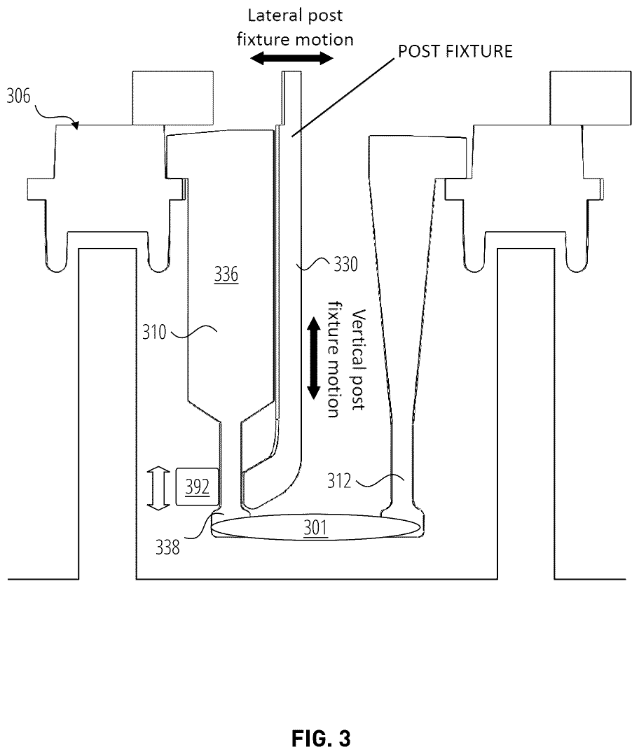

The elongate tissue fixture limiter 130 also has a distal end extending into the well 108 in between the flexible post 110 and the rigid post 112. However, the distal end of the tissue fixture limiter 130 does not extend directly between the distal ends of the flexible post 110 and rigid post 112, so as not to contact any tissue specimen attached therebetween. As shown, an upper portion of tissue fixture limiter 130 abuts the upper portion 136 of the flexible post 110, but does not contact the distal portion 138. Thus, the flexible distal portion 138 of the flexible post 110 can deflect toward the rigid post 112 (e.g., from a contraction force of the tissue specimen) until it makes contact with the tissue fixture limiter 130. At that point, the distal portion 138 of the flexible post 110 cannot deflect toward the rigid post 112 any further. In effect, the tissue fixture limiter 130 increases the stiffness of the flexible post 110, selectively converting it to a rigid post or a near-rigid post. This increases the maximum allowable stretch of the tissue specimen utilizing the tissue fixture modulating apparatus 106, which causes the rigid post 112 to move away from the flexible post 110 as described below. In any embodiment, the tissue fixture limiter 130 may be configured to contact one or more of the first tissue fixtures at different locations in order to modulate the stiffening effect. Any embodiment may include one, two, or more tissue fixture limiters for each tissue fixture in each well. One such example which may be adapted to the tissue modulating system 100 is shown in FIG. 3.

An aperture 142 is positioned directly over each well to facilitate observation of the tissue specimen therein. Such aperture 142 includes a series of aligned apertures extending through all structure otherwise obstructing a top-down view of the well 108, including apertures extending through the actuator lid 132, printed circuit board 134, and any other lid.

FIG. 1E and FIG. 1F show aspects of the tissue fixture modulating apparatus 106.

FIG. 1E shows a pivoting beam 120 pivotally coupled with a pivot point 118 at either end. Pivot points 118 may be modular elements attachable to the multi-well plate 104 or intermediate structure such as the well plate lid 114 or actuator lid 132. In other embodiments, pivot points 118 are integrally formed with one or more of said structure(s).

As shown clearly in FIG. 1F (wherein the fixture bridge 128 and post beam 116 are hidden), a lever forming part of an arm actuator 124 extends away from the pivoting beam 120. The arm actuator 124 has a receiver 144 at an end thereof which is coupled to or configured to contact an actuator 146 (shown schematically), for example a linear actuator or rotary actuator, which may be manual (e.g., a manual lead screw adapted for manual adjustment) or electrical (e.g., stepper motors and servos). In some embodiments, the actuation assembly 122 includes the actuator 146. Advantageously, movement of the arm actuator 124 causes all rigid posts 112 to pivot or rotate about the pivot point 118. The arm actuator 124 may extend away from the pivoting beam 120 in any direction, depending on device design.

In other embodiments, the tissue fixture modulating apparatus 106 acts on the rigid posts 112 in a different manner and does not include an arm actuator 124. For example, in some embodiments, the tissue fixture modulating apparatus 106 comprises an actuator (e.g., a servo or stepper motor) operably coupled to an end of the pivoting beam 120 (i.e., with its rotational axis parallel to the longitudinal direction of the pivoting beam 120), thus directly turning the pivoting beam 120 about the pivot point 118. In other embodiments, the tissue fixture modulating apparatus 106 comprises a linear actuator that directly acts on at least one of the rigid posts 112 (i.e., using the rigid post 112 as a lever arm in order to rotate the pivoting beam 120 about the pivot point). In still other embodiments, the tissue fixture modulating apparatus 106 includes a linear actuator operably coupled to translate the pivoting beam 120 along a track (rather than rotate about the pivot point 118). In still other embodiments, the tissue fixture modulating apparatus 106 comprises one or more magnets (e.g., electromagnets) which selectively magnetically attract and/or repel another element of the system (e.g., a magnet disposed in one or more of the tissue fixtures).

In any embodiment, the actuator 146 may be operably coupled to the pivoting beam 120 directly or indirectly, e.g., via a gear train, cam, rocker arm, linkage, or other intermediate structure.

Returning to FIG. 1E, fixture bridge 128 comprises a base 148 from which the tissue fixture limiters 130 extend away on a first side, and from which securement structure such as pins 140 extend away on a second side. The pins 140 are sized and positioned to be received within apertures of the surrounding enclosure, such as actuator lid 132 and/or printed circuit board 134. Cutouts 150, 152 are formed in the base 148 such that the fixture bridge 128 assembles over and accommodates pivotal motion of the arm actuator 124.

Turning again to FIG. 1F, the pivoting beam 120 includes a plurality of stops. A maximum and minimum stretch limit are established via the receiver 144 and a baseline stop 154, respectively. The receiver 144 limits the maximum allowable stretch by contacting the well plate lid 114. The stop 154 establishes a baseline unstretched position. The pivoting beam 120, and thereby a tissue specimen, may be biased towards the minimum unstretched limit via a spring or other return mechanism which may be disposed in the stop 154 or another stop 156. The receiver 144, stop 154, and stop 156 include calibrating apparatuses, e.g., set screws. Accordingly, the actuation assembly 122 can be calibrated during setup.

Turning to FIG. 1G, embodiments of the tissue modulating system 100 may comprise computing elements in addition to (or as part of the tissue modulating device 102). Such elements include a processor 158 and a non-transitory computer-readable storage medium such as a virtual processor memory 160, physical processor memory 162, or persistent secondary storage 164. Such storage medium(s) stores instructions that when executed by the processor 158, cause the processor to perform any one or more of the following: execute a tissue modulating protocol according to any of the methods described below; actuate a tissue fixture modulating apparatus; actuate an actuation assembly; or apply a voltage to one or more electrodes.

The tissue modulating system 100 may optionally comprise an input device adapter 166, an output device adapter 168, a network interface adapter 170, a bus 172, a virtual processor memory 160, an input device 174, and an output device 176. The virtual processor memory 160 may comprise a physical processor memory 162, an operating system 178, instructions 180 (e.g., applications storing instructions for executing one or more methods described herein), and other libraries and subsystems 182. Part of the persistent secondary storage 164 and/or the bus 172 may be comprised by the tissue modulating device 102 and/or the virtual processor memory 160.

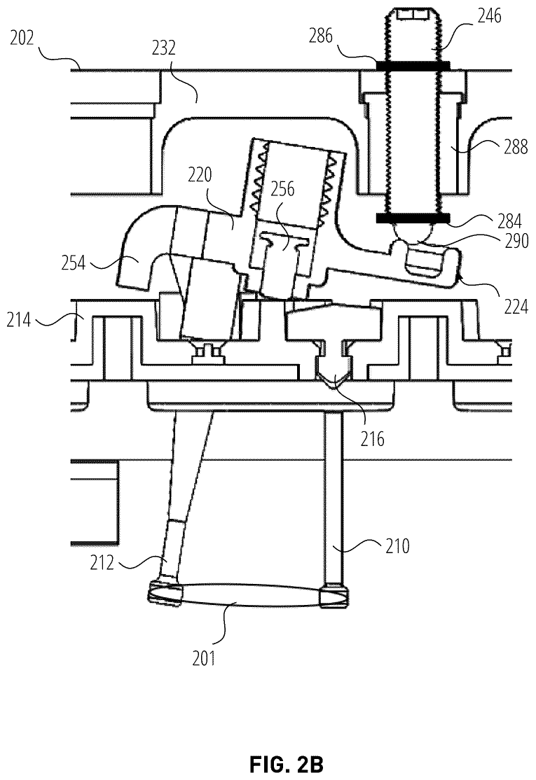

FIG. 2A-FIG. 2B respectively show a tissue fixture modulating apparatus 206 in a first state (i.e., a default state) and a second state (i.e., a modulated state or deflected state) in order to demonstrate operation and effect on a tissue specimen 201. One purpose of the tissue fixture modulating apparatus 206 is to statically stretch tissue specimens via a known displacement of one or both of the tissue fixtures.

The tissue fixture modulating apparatus 206 is a manually actuated embodiment. However, the described structure may be readily adapted to other embodiments with an automated actuator, e.g., a servo motor, a stepper motor, a linear actuator, etc.

The tissue modulating device 202 and tissue fixture modulating apparatus 206 include similar elements as the tissue modulating device 102 and tissue fixture modulating apparatus 106 of FIG. 1A-FIG. 1F. Accordingly, such elements will not be reintroduced.

In the first state of FIG. 2A, the tissue fixture modulating apparatus 206 is calibrated such that the tissue specimen 201 is in an un-actuated state (e.g., the tissue specimen 201 is unloaded and un-stretched and has a first length). The calibration system includes an actuator 246 (here a manual lead screw with travel limits defined by stops 284, 286) and stops 254, 256. To calibrate the tissue fixture modulating apparatus 206, actuator 246 is retracted until stop 284 contacts a threaded collar 288 press-fit within an aperture of the actuator lid 232. In such position, an effector 290 of the actuator 246 is positioned adjacent to arm actuator 224, but does not exert sufficient force thereon to cause the pivoting beam 220 to rotate (clockwise in this figure) about a pivot point. Some embodiments of the actuator 246 include a scale that indicates movement of the rigid post 212.

A clearance between the effector 290 and actuator 246 is further adjustable via stop 256, which is disposed on the pivoting beam 220 and indicates the maximum stretch of the tissue specimen 201 and indicates when stretch has been initiated on the tissue specimen 201. Similarly, stop 254 limits counterclockwise rotation of the pivoting beam 220. The tissue fixture modulating apparatus 206 may be biased toward this un-actuated state via a spring or other return device. In the un-actuated state, the tissue specimen 201 is unstretched.

The second state of FIG. 2B illustrates the tissue fixture modulating apparatus 206 in a second state or actuated state such that the tissue specimen 201 has a longer second length. The actuator 246 is rotated such that the effector 290 enacts a force on arm actuator 224, causing pivoting beam 220 to pivot about a pivot point. Accordingly, the rigid post 212 rotates away from the flexible post 210 by a known distance correlated to the displacement of the effector 290, causing the tissue specimen 201 attached therebetween to stretch relative to its length when the tissue fixture modulating apparatus 206 is in the first state of FIG. 2A.

The stretch (e.g., percentage elongation) of the tissue specimen 201 can be determined via a sensing system, e.g., a) utilizing one or more magnetometers that sense the absolute position of a magnet embedded in the distal end of the flexible post 210 and/or rigid post 212, b) a camera-based optical system that tracks the centroid to centroid distance of the distal ends of the flexible post 210 and rigid post 212. Additionally, the tissue fixture modulating apparatus 206 can be operated in an open-loop configuration, e.g., without stretch measurement, for example with assays wherein rigid post displacement, in combination with a flexible post or not, is adequate.

As will be appreciated from the description of the tissue modulating protocols below, stretching the tissue specimen 201 can stretch the tissue specimen 201 when the tissue specimen 201 is contracting (naturally or from stimulation current), or even induce tissue injury.

When the tissue specimen 201 is attached between the rigid post 212 and flexible post 210, the distal end of the flexible post 210 will deflect by some amount when the rigid post 212 is displaced under the operation of the tissue fixture modulating apparatus 206. Indeed, deflection of the distal end of the flexible post 210 enables computation of tissue characteristics, e.g., contractile force measurements based on beam theory. Accordingly, the tissue fixture modulating apparatuses described herein are all means for displacing the flexible post (directly or indirectly).

It is advantageous to directly actuate (e.g., displace) the rigid post 212 rather than directly actuate the flexible post 210 because there is greater certainty that a given input to the tissue fixture modulating apparatus will displace the rigid post by a known distance, whereas the flexibility of the flexible post can introduce variation in the correlation between input and output. Restated, a known input to the actuator 246 creates a highly predictable and repeatable movement of the rigid post 212.

Tissue modulating systems of the present disclosure include variants of the specific embodiments described. For example, some tissue modulating systems include one tissue fixture modulating apparatus per well. Such embodiments would include a greater number of actuators for greater customization between wells. In other embodiments, the tissue modulating systems include a single tissue fixture modulating apparatus for all wells, e.g., for greater economy.

The tissue fixture modulating apparatuses and actuation assemblies described herein may be adapted to couple with a multi-well plate, e.g., as part of a consumable product. Specifically, at least part of the tissue fixture modulating apparatus may be integrated with a well plate lid (e.g., actuator lid 132) and/or a stimulation plate that couples to a well plate; directly integrated with a well plate, well plate lid, or stimulation lid; or may be a separate apparatus that couples to a well plate lid, stimulation plate, and/or well plate.

The tissue fixture modulating apparatuses of FIG. 1A-FIG. 2B are manually adjusted embodiments; however, variants of all such embodiments utilize electronic, pneumatic, magnetic, or other actuators, e.g., wherein one or more of the actuators include electronic steppers motors and/or servos. Advantageously, electronic actuators enable tissue modulating protocols that impart well-controlled strain rates and/or a strain rates not achievable with manual actuators.

Advantageously, the tissue modulating systems described herein enable computation of tissue characteristics based upon beam theory equations resulting from the known geometry and properties of the flexible post and the known elongation of the tissue, whereas known systems cannot obtain this data simultaneously.

For example, according to one representative method, the tissue modulating system models the flexible post 210 as a cantilever beam and determines tissue characteristics of the tissue specimen 201 based upon the representative equation shown below:

@x=L, where x is a location of perpendicular force on a beam

where δₘₐₓ is the maximum displacement of the distal end of flexible post 210, F is the force acting on the flexible post 210 (and thus the strength of the tissue specimen 201), L is the length of the flexible post 210, E is the elastic modulus of the flexible post 210 (e.g., Young's Modulus), and I is the second moment of area of the cross section of the flexible post 210.

Tissue modulating systems may include control systems (e.g., control system 184) including, for example, one or more sensor arrays that track movement of the tissue fixtures and tissue specimens. The control systems are operably connected to the processor and tissue fixture modulating apparatus (particularly the actuation assembly) in order for temporal modulation of the tissue fixtures.

In some embodiments, the control system includes one or more cameras that optically track tissue fixture limiter position. In such embodiments, the distal ends of the flexible posts and/or rigid posts may be colored or have fiducial markers that facilitate imaging. In other embodiments, the control system includes a plurality of magnetometers and magnets. In such embodiments, one or more magnetometers is positioned proximal to the distal end of each flexible post, which has a magnet embedded therein or attached thereto.

Parameters which may be controlled simultaneously or individually, for individual well or for a plurality of wells include tissue specimen output parameters including tissue stretch (e.g., passive stretch), strain, contraction force, twitch force, muscle fiber recruitment, dwell time, peak displacement, stimulation current, and/or other parameters. To control any one or more of the foregoing parameters, the control system may vary one or more input parameters including a stimulation electrode voltage, stimulation current level, stimulation current timing, stimulation input waveform, an input to an actuation assembly of a tissue fixture modulating apparatus, a position and/or movement of one or more tissue fixtures.

Based on feedback from the control system 184, the tissue modulating system 100 may modulate one or more inputs based on feedback from real-time measurement of functional outputs for one or more tissue specimens over time. Such feedback may be implemented independently for each well of a plurality of wells, regionally (for a subset of wells), or globally (i.e., for all wells).

Control systems may include one or more control modes. In a semi-real-time control mode, the control system determines an “average max displacement” of the one or more tissue fixtures over N cycles as feedback.

In a real-time mode, the control system allows for timing the tissue fixture actuation to avoid eccentric contraction of the tissue specimen and/or simulate Pressure-Volume relationships of cardiac tissue. In some such embodiments, a sensor array determines a contraction of an autonomously beating cardiac tissue specimen, or stimulation electrodes apply safe and non-injurious electrical stimuli which cause cardiac tissue to contract with a known timing and frequency. Then, as the tissue specimen relaxes, the control system quickly actuates one or more tissue fixtures to stretch the tissue specimen using an appropriate waveform that reaches peak stretch displacement and then returns quickly to baseline (original passive tension distance) at a rate equal to or slightly faster than tissue contraction speed. Additionally, this allows for accurately converting the actuator position into tissue length. Due to the viscoelastic nature of the flexible post and/or the tissue specimen, a user-defined time trace of the actuator position will not be identical to the resulting time trace of the tissue specimen length. Furthermore, if the tissue specimen and/or flexible post properties change over time (e.g., due to mechanical creep or relaxation), the resulting time trace of the tissue specimen length will change over time even if the user-defined time trace of the actuator position is unchanged. The sensor array-based control system measures the tissue specimen length and adapts the actuator position to achieve the desired tissue specimen length. Such an implementation also enables the user to directly input the desirable time trace of the tissue specimen length instead of the actuator position as the indirect input.

In an “intelligent” mode, the control system facilitates tissue specimen maturation via stretch regimen and periodic tissue specimen contraction strength measurements, e.g., stretch for N cycles then stop stretching for a time period to make a contraction force measurement via flexible post beam mechanics and optical/magnetic tracking algorithms.

The control system may utilize machine learning algorithms to optimize stretching via different stretching variables on different wells with different variables, e.g., stretch waveform changes such as dwell time, peak displacement, asymmetry of sinusoid, etc.

Generally, the control system may be adapted to independently control tissue fixture modulation and tissue specimen stretch in different wells of the tissue modulating system.

FIG. 3 shows aspects of another tissue fixture modulating apparatus 306 in order to illustrate an alternative tissue fixture limiter 330. Consistent with the tissue fixture limiters previously described, the tissue fixture limiter 330 is a rigid object (e.g., a beam) and is in physical contact with the flexible post 310. The flexible post 310 has a bent distal end that closely abuts or contacts the distal portion 338 of the flexible post 310.

The tissue fixture limiter 330 is movable between a fully deployed position and a fully retracted position. In the fully deployed position, the tissue fixture limiter 330 is positioned to intercept the flexible post 310 near the distal portion 338 thereof as the distal portion 338 moves toward the rigid post 312. In the fully retracted position, the tissue fixture limiter 330 is moved away from the distal portion 338 of the flexible post 310 to the fullest extent permitted by the tissue fixture modulating apparatus 306. In some embodiments, the tissue fixture limiter 330 may still be configured to intercept the flexible post 310 in the fully retracted position, but at a position further away from the distal portion 338 as compared to the fully deployed position. In some embodiments, the tissue fixture limiter 330 may be configured to not intercept the flexible post 310 at all. The tissue fixture limiter 330 may be secured at any position between the fully deployed position and the fully retracted position.

In the deployed position, the tissue fixture limiter 330 is moved along the length of the flexible post 310 such that the distal end of the tissue fixture limiter 330 no longer abuts the distal end of the flexible post 310 (although it may abut the flexible post 310 if the flexible post 310 is bent toward the rigid post 312). Accordingly, the tissue fixture limiter 330 permits the deflection of the flexible post 310 toward the rigid post 312 in the retracted position.

In the deployed position shown in FIG. 3, the distal end of the tissue fixture limiter 330 abuts the distal end of the flexible post 310, thus preventing its movement in a first direction toward the rigid post 312 when the tissue specimen 301 contracts. In the deployed position, the distal end of the tissue fixture limiter 330 may directly contact or be spaced apart from the distal end of the flexible post 310 as described below. When the flexible post 310 is unable to bend, and the tissue specimen 301 enters isometric contraction (i.e., isovolumetric contraction), resulting in several possible modalities:

For example, the tissue fixture limiter 330 may be configured such that in the deployed position, its distal end is spaced apart from the distal end of the flexible post 310 by a prespecified distance. Therefore, when the tissue specimen 301 contracts and begins to shorten, the flexible post 310 is permitted to bend by the prespecified distance. Therefore, the tissue goes into a delayed isometric contraction.

As another example, the tissue fixture limiter 330 can be moved between the deployed and retracted positions dynamically, e.g., to allow the tissue specimen 301 to exit the isometric contraction and continue shortening as it continues to contract.

In some embodiments, an optional secondary tissue fixture limiter 392 on the opposite side of the flexible post 310 prevents the flexible post 310 from moving in a second direction, i.e., returning to a less bent position. In an exemplary use case, this secondary tissue fixture limiter 392 forces the tissue specimen 301 into isometric relaxation, and it can be moved dynamically. In another exemplary use case, the secondary tissue fixture limiter 392 limits a length of the tissue specimen 301 in a relaxed state. In any embodiment, the secondary tissue fixture limiter 392 may be secured at any position between and including a fully deployed position and a fully retracted position.

In any embodiment, each tissue fixture limiter is independently movable between the fully retracted position and the fully deployed position. In any embodiment, a plurality of tissue fixture limiters is collectively movable between the fully retracted position and the fully deployed position, e.g., under common control.

The tissue fixture limiter 330 is representative and not limiting. For example, in some embodiments, the tissue fixture limiter is inserted into a cavity within the flexible post by a variable depth to vary the stiffness of the flexible post. Some embodiments include one or two tissue fixture limiters for both the first tissue fixture and second tissue fixture in each well, e.g., to enable different isometric contraction protocols. In some embodiments, a single tissue fixture limiter obstructs a travel path of two or more tissue fixtures in a single well.

The dynamic and bidirectional nature of the tissue fixture limiters described herein give rise to various temporal force-length patterns, and such patterns can replicate patterns observed in vivo.

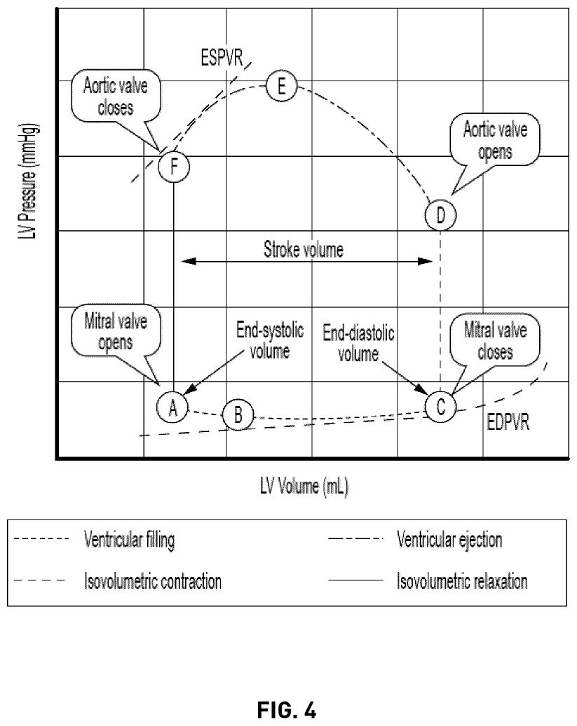

FIG. 4 illustrates a pressure-volume relationship of a heart which can be simulated according to a tissue modulating protocol and a method of using tissue modulating systems as described herein. By combining two tissue fixture limiters as shown in FIG. 3, one can see how the pressure-volume curves of a heart can be replicated. In the context of a tissue modulating system as described herein, the analogues of pressure and volume are force and length, respectively.

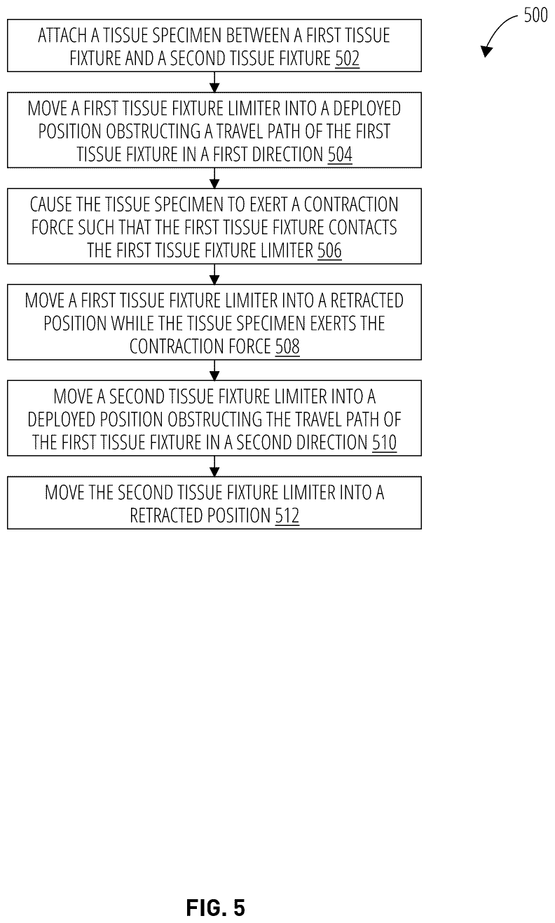

FIG. 5 illustrates a method of modulating a tissue specimen which simulates the pressure-volume relationship of a heart (shown in FIG. 4). The following method is described with reference to the tissue fixture modulating apparatus 306 of FIG. 3, but can be performed independently.

In step 502, method 500 attaches a tissue specimen between a first tissue fixture and a second tissue fixture.

In step 504, method 500 moves a first tissue fixture limiter into a deployed position obstructing a travel path of the first tissue fixture in a first direction.

In step 506 corresponding to (C) in FIG. 4, method 500 causes the tissue specimen to exert a contraction force such that the first tissue fixture contacts the first tissue fixture limiter. A short time period thereafter (e.g., hundredths or tenths of a second), the first tissue fixture contacts the tissue fixture limiter 330 and the tissue specimen 301 enters isometric contraction. Accordingly, steps 502-506 exemplify an isometric contraction protocol of a tissue specimen.

In step 508, method 500 moves a first tissue fixture limiter into a retracted position (e.g., a partially or fully retracted position) while the tissue specimen exerts the contraction force. The tissue specimen 301 shortens until reaching a maximum contraction at (F) in FIG. 4.

In step 510, method 500 moves a second tissue fixture limiter into a deployed position obstructing the travel path of the first tissue fixture in a second direction. The tissue specimen 301 relaxes, causing the first tissue fixture to contact the secondary tissue fixture limiter 392, e.g., holding the distal portion 338 in a deflected position and the tissue specimen 301 enters isometric relaxation.

In step 512 corresponding to (A) in FIG. 4, method 500 moves the second tissue fixture limiter into a retracted position (e.g., a partially or fully retracted position).

The tissue fixture limiter may also be used to control the effective stiffness of the flexible post. For example, referring back to FIG. 3, the tissue fixture limiter 330 is in contact with or at a prespecified distance from the flexible post 310, and the tissue fixture limiter 330 is moved vertically and closer to the base of the flexible post 310. The tissue specimen 301 attached between the flexible post 310 and rigid post 312 contracts or the distal portion 338 of the flexible post 310 is deflected away from the upper portion 336. In either case, the distal portion 338 bends in the direction of the rigid post 312. Upon deflection of the distal portion 338, the tissue fixture limiter 330 and the flexible post 310 eventually come in contract. A tissue fixture limiter 330 positioned closer to the upper portion 336 of the flexible post 310 will block the deflection of a part of the distal portion 338, therefore allowing for the partial deflection of the distal portion 338.

Compared to a case where the tissue fixture limiter 330 is completely retracted and the flexible post 310 bends freely, the partly deflected flexible post 310 features a greater stiffness. Therefore, while the distal portion 338 can be displaced, the amount of force the flexible post 310 exerts on the tissue specimen 301 increases as the distal end of the tissue fixture limiter 330 is moved toward the distal portion 338 of the flexible post 310. Accordingly, the effective stiffness of the flexible post 310 can be modulated by the vertical position of the tissue fixture limiter 330. The same effect can be achieved using any tissue fixture limiter described herein.

Modulating the vertical position of the tissue fixture limiter modulates the effective post stiffness and enables modeling an increasing or decreasing load. This is particularly relevant when modeling the load against which the tissue specimen is contracting. For example, the variable positioning of the tissue fixture limiter can be used to control the afterload features of FIG. 3. In the case of the heart, the slope of the curve DE represents the fluid resistance of the aortic valve. The pathological narrowing of the aortic valve (such as in aortic stenosis) leads to the increased resistance of the valve and gradually leads to the structural and functional remodeling of the heart.

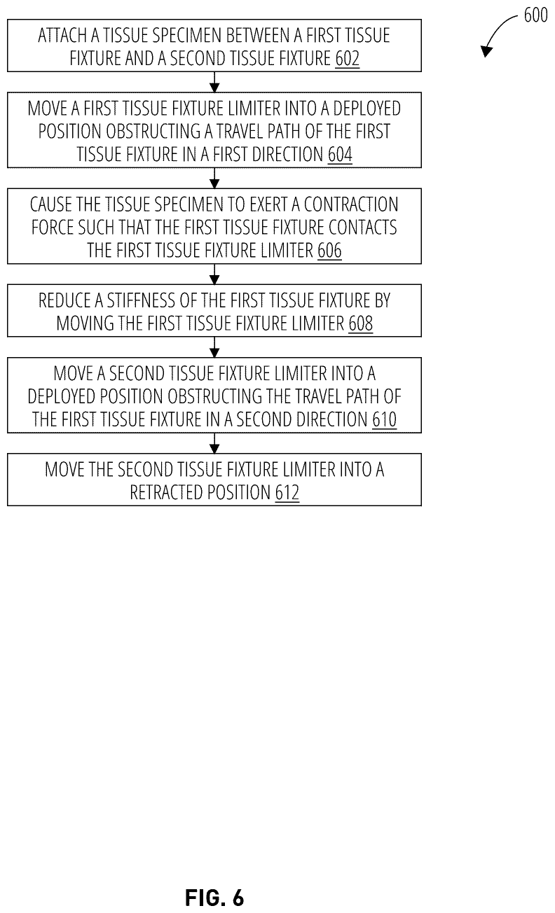

FIG. 6 illustrates another method of modulating a tissue specimen, which simulates the pressure-volume relationship of a heart (shown in FIG. 4) utilizing a tissue modulating apparatus. The following method is described with reference to the tissue fixture modulating apparatus 306 of FIG. 3, but may be performed independently.

In step 602, method 600 attaches a tissue specimen between a first tissue fixture and a second tissue fixture. Referring to FIG. 3, the tissue specimen 301 is attached between flexible post 310 and rigid post 312.

In step 604, method 600 moves a first tissue fixture limiter into a deployed position obstructing a travel path of the first tissue fixture in a first direction. Referring to FIG. 3, the tissue fixture limiter 330 is moved into the deployed position shown.

In step 606, method 600 causes the tissue specimen to exert a contraction force such that the first tissue fixture contacts the first tissue fixture limiter, thus increasing the effective stiffness of the first tissue fixture. Referring to FIG. 3 and FIG. 4 together, the tissue specimen 301 contraction starts at (C) and in a first time period, the flexible post 310 hits the tissue fixture limiter 330 at (C), wherein the vertical position of the distal end of the tissue fixture limiter 330 immobilizes the flexible post 310 and the tissue specimen 301 enters isometric contraction.

In step 608, method 600 reduces the stiffness of the first tissue fixture (relative to step 606) by moving the first tissue fixture limiter. Referring to FIG. 3 and FIG. 4, at (D), while the tissue specimen 301 is still exerting a contraction force, the first tissue fixture limiter 330 is moved towards the upper portion 336 of the flexible post 310 and the tissue specimen 301 starts to shorten.

In step 610, method 600 moves a second tissue fixture limiter into a fully or partially deployed position obstructing the travel path of the first tissue fixture in a second direction. Referring to FIG. 3 and FIG. 4, once the tissue specimen 301 reaches its maximum contraction at (F), the secondary tissue fixture limiter 392 approaches and comes in contact with the flexible post 310. During the relaxation, the tissue specimen 301 relaxes but the secondary tissue fixture limiter 392 blocks the flexible post 310 and so the tissue specimen 301 enters isometric relaxation.

In step 612, method 600 moves the second tissue fixture limiter into a partially or fully retracted position. Referring to FIG. 3 and FIG. 4, the secondary tissue fixture limiter 392 is moved out of the way at (A), the tissue specimen 301 continues to relax and lengthen until the next contraction starts at (C).

The tissue fixture limiter may also be used to increase the maximum amount of stretch the system can cause to the tissue, independently of the tissue stiffness. Such use of the fixture is particularly relevant in eccentric injury protocols, where the tissue stiffness is expected to increase three orders of magnitude during contraction and therefore the tissue will resist stretching. In an embodiment of the eccentric injury protocol described below, the tissue specimen is electrically stimulated to initiate a contraction, the tissue fixture limiter is introduced to immobilize the flexible post, and the actuator displaces the rigid post to stretch the tissue. In the presence of the tissue fixture limiter, the rigid post displacement will be equal to the tissue stretch. In the absence of the fixture, the rigid post displacement will mainly translate into the displacement of the flexible post and therefore the tissue stretch will be significantly decreased.

Assuming the tissue specimen is not contracting, but the tissue fixture limiters are in place and the posts are moved utilizing tissue fixture modulating apparatuses, it is possible to forcefully change (instead of passively freezing) the length of the tissue to either higher or shorter lengths. Such length changes can be used to model events happening in skeletal muscle, and most interestingly to model muscle injury/strain.

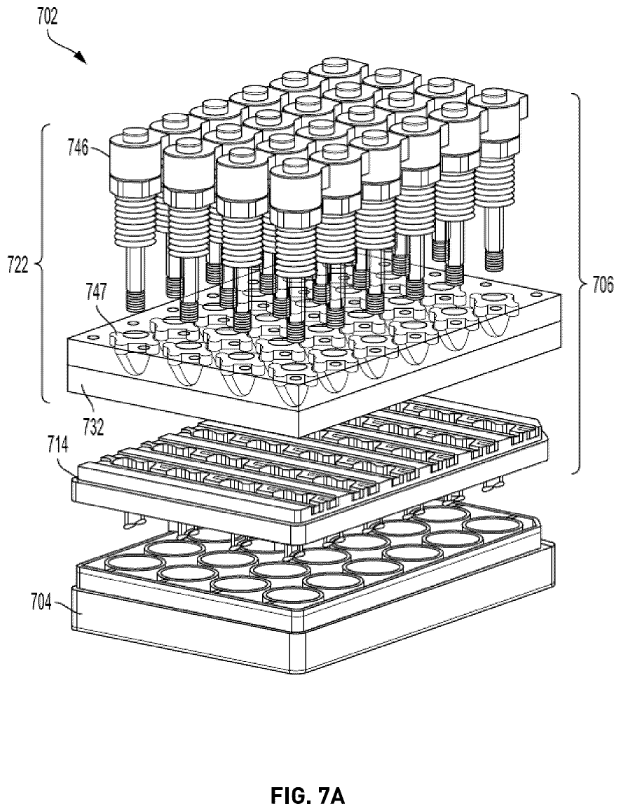

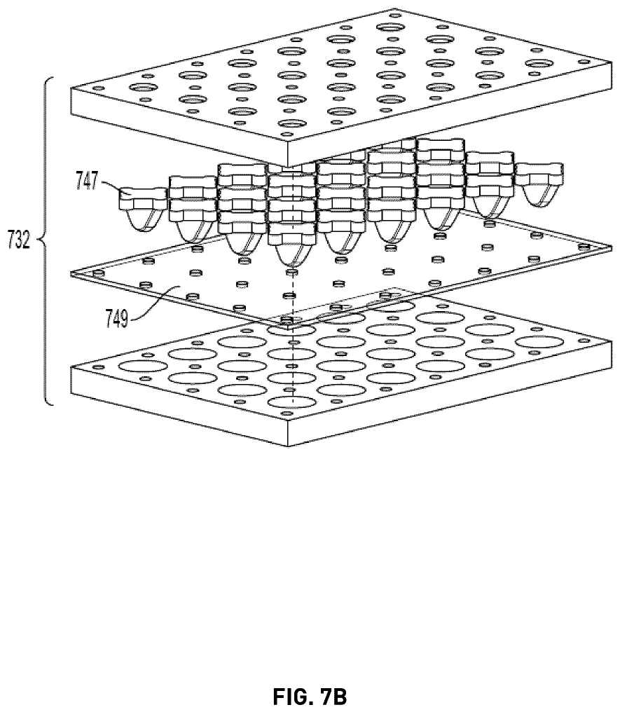

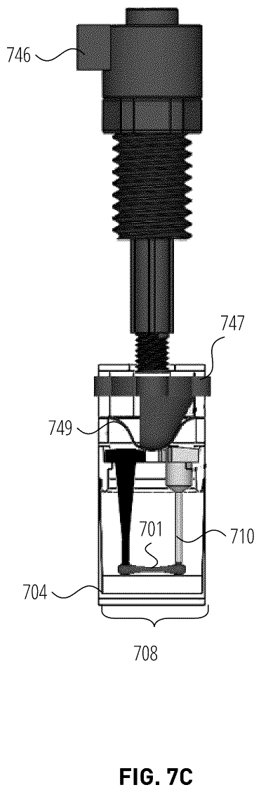

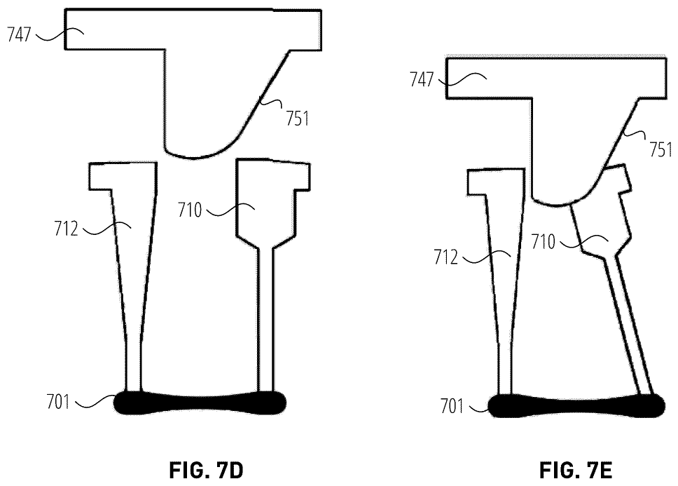

FIG. 7A-FIG. 7E shows aspects of another representative tissue modulating device 702 comprising a tissue fixture modulating apparatus 706 and actuation assembly 722.

Referring to FIG. 7A, the tissue modulating device 702 includes a lid assembly comprising a well plate lid 714 formed as a lattice integrated with an array of post pairs, each post pair having at least a rigid post and a flexible post. In this embodiment, rigid posts and flexible posts form part of the lid assembly. However, in this embodiment and others described herein, the rigid posts and/or flexible posts may also be considered part of the tissue fixture modulating apparatus 706. The lid assembly is attachable to a multi-well plate 704 such that each post pair extends into one well.

The tissue fixture modulating apparatus 706 includes an actuation assembly 722 having an array of actuators 746 (e.g., linear actuators) operably connected to an actuator lid 732 with an array of pistons 747.

Each piston 747 is operably coupled to an actuator 746 that drives the piston 747 down to stretch the tissue specimen attached between the post pair between an unstretched state (see FIG. 7D) and a stretched state (see FIG. 7E). As shown most clearly in FIG. 7D and FIG. 7E, the piston 747 has a chamfered end configured to push down an upper edge of the flexible post 710, providing linear stretching while allowing for minor misalignment between the piston 747 and the flexible post 710.

In the embodiment shown, the actuator lid 732 includes one piston 747 for each well. Other embodiments may include fewer pistons than wells by utilizing a linkage to actuate a plurality of wells with each piston. One or more of the pistons 747 may have a return mechanism (e.g., spring) which returns the respective piston to a default state corresponding to an unstretched state of the tissue specimen.

Turning to the exploded view of FIG. 7B, the actuator lid 732 optionally includes a flexible silicone sheet or gasket 749 sandwiched between a lower lid shell and the array of pistons 747 and an upper lid shell to prevent biological contamination between the actuation assembly 722 and the wells.

The actuation assembly 722 is designed to prevent any stretching “cross-talk” in the adjacent wells when the top edge of a flexible post positioned between the adjacent wells is compressed. In some embodiments, the array of pistons 747 can be configured to boss press or otherwise individually secured to the actuator lid 732 to prevent “cross-talk” between adjacent wells.

A spring force interface can be provided between the array of actuators 746 and the well plate lid 714 to provide vertical force for facilitating an automatic return of the angled pistons 747 upon disengagement from the respective actuators 746. A quick-connect interface such as canted coil springs or z-axis linear actuator can provide an initial contact between the actuator 746 and the underlying angled piston 747 without applying an undue level of initial force on the flexible posts.

The tissue modulating device 702 shown in FIG. 7A is illustrated in a 24-well configuration. Other embodiments can be adapted for any multi-well configuration having a smaller or larger number of wells. As the forces needed to stretch engineered tissues can be very small, higher throughput apparatuses (such as a 96-well system) can be made with miniaturization of the linear actuators.

FIG. 7C is a cross-sectional view of an aspect of the tissue fixture modulating apparatus 706 of FIG. 7A-FIG. 7B. As previously described, the actuator 746 is positioned above a well 708 of the multi-well plate 704. The actuator 746 is rotationally driven to engage with the angled piston 747 to cause rotation of a flexible post 710 and thereby induce incremental stretching of a tissue specimen 701 attached thereto. Each piston 747 has fine resolution for controlling a precise amount of linear motion and can be configured to push down, via the angled piston 747, the top edge of the flexible post 710, to induce stretching.

The tissue modulating device 702 illustrates important variations which are adaptable to tissue modulating systems of tissue modulating devices of the present disclosure. First, the tissue modulating device 702 illustrates the principle of directly actuating each well with a dedicated actuation assembly 722, i.e., one actuation assembly per well. Second, the tissue modulating device 702 illustrates the principle of actuating the flexible post of a post pair. Both principles may be adapted to other embodiments not expressly described herein. For example, a variant of the tissue modulating device 102 of FIG. 1A-FIG. 1G may utilize one actuator 146 per well 108 (rather than utilizing one actuator 146 to actuate a plurality of wells). As another example, a variant of the tissue modulating device 102 of FIG. 1A-FIG. 1G may actuate the flexible post 110 of each well, rather than the rigid post 112.

Any tissue modulating system or tissue modulating device described herein can be further improved with integration of an electrical stimulation apparatus such as one or more electrodes operably coupled with stimulation circuitry, e.g., the stimulation printed circuit board 134 of FIG. 1A-FIG. 1E, and other computing elements of the tissue modulating system. The electrodes and stimulation circuitry may be integrated with a well plate lid as a modular stimulation lid that can be readily coupled with a multi-well plate and tissue modulating apparatus. In any embodiment, the tissue modulating apparatus may include some or all components of the stimulation apparatus.

FIG. 8 shows a cross-sectional view of an aspect of a tissue modulating device which is identical to the tissue modulating device 702 of FIG. 7A-FIG. 7E, with the addition of stimulation circuitry 834 operatively connected to one or more stimulation electrodes 835 extending into the well. The electrode 835 is offset from the rigid post and the flexible post by a distance, e.g., about 3 mm to about 10 mm.

In use, a tissue specimen is attached between the rigid post and the flexible post in a medium. A processor executes tissue modulating protocol instructions stored in the computer-readable storage medium, causing the stimulation circuitry 834 to apply a voltage to the electrode 835, which in turn stimulates the tissue specimen with a stimulation current. The stimulation circuitry 834 can be adapted to provide different stimulation current amplitudes and waveforms to the tissue specimen, depending on the tissue modulating protocol.

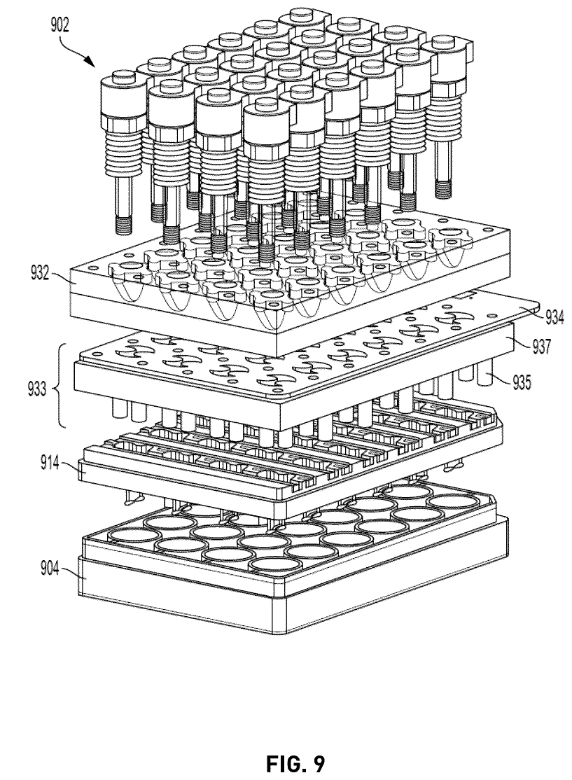

FIG. 9 illustrates one representative structure for integrating an electrical stimulation apparatus with any tissue modulating device of the present disclosure, e.g., to produce individually controllable and/or simultaneous or synchronized electrical stimulation of multiple tissue specimens at any specified times during individual stretching cycles of multiple tissues.

The tissue modulating device 902 is similar to the tissue modulating device 702 of FIG. 7A-FIG. 7E, with the addition of a stimulation apparatus 933 (collectively, the “electrode lid assembly” or stimulation lid or stimulation plate) comprising a plurality of electrodes 935 operatively coupled with a stimulation lid 937 and a printed circuit board comprising stimulation circuitry 934.

In this representative embodiment, the stimulation apparatus 933 is interposed between the actuator lid 932 and the well plate lid 914. In other embodiments, the stimulation apparatus 933 is integrated with the actuator lid 932 and/or the well plate lid 914 (e.g., a monolithic structure). In still other embodiments, the stimulation apparatus 933 is configured for direct attachment to the multi-well plate 904, i.e., interposed between the multi-well plate 904 and the well plate lid 914 such that the post pairs extend through the stimulation apparatus 933 and into the wells. The stimulation apparatus 933 has apertures therethrough which are configured to receive the actuators of the actuator lid 932 (e.g., the angled pistons).

In some embodiments, the stimulation circuitry 934 is configured to apply a different stimulation profile to each electrode, e.g., stimulation profile being characterized by one or more of a voltage, a waveform, or a stimulation time. In other embodiments, the stimulation circuitry 934 is configured to apply a common stimulation profile to a plurality of wells.

The tissue modulating systems are adapted to modulate or condition tissue specimens according to tissue modulating protocols, for example an eccentric strain protocol, an eccentric stretch during fatigue protocol, an eccentric stretch with partial recruitment protocol, a high strain during relaxation protocol, a repetitive high rate strain no stimulation protocol, an isometric contraction protocol, a stiffness protocol, or a force-length protocol. Such methods and protocols will now be described. Any such tissue modulating protocol may be repeated, e.g., for N cycles, optionally followed by a measurement of a tissue characteristic (e.g., tissue stiffness).

Such tissue modulating protocols may be performed on tissue specimens independently of the tissue modulating systems described herein. To clarify, the tissue modulating protocols described herein may be implemented on additional systems and devices other than those described herein. Any of the tissue modulating protocols described herein may be implemented as instructions stored on a computer-readable storage medium of a tissue modulating system. Such tissue modulating systems may be programmed to implement the same tissue modulating protocol on a plurality of wells (e.g., all wells of a multi-well plate) and/or different tissue modulating protocols on different wells contemporaneously.

The tissue modulating protocols described below may be performed alone or as part of a larger method, e.g., as part of a method of modulating a tissue specimen, a method of determining a tissue characteristic, or a method of operating a tissue modulating device.

Certain of the tissue modulating protocols are designed to induce maturation or injury or damage to the tissue specimen (particularly tissue specimens comprising skeletal muscle and cardiac muscle), whereas other tissue modulating protocols are designed to measure stiffness, force-length relationship, or other tissue characteristics.

The following tissue modulating protocols may be applied to one or more tissue specimens attached between a first tissue fixture (e.g., a rigid post) and a second tissue (e.g., a flexible post) in a tissue fixture modulating apparatus, wherein the tissue specimen is electrically coupled to means for electrical stimulus such as a stimulation plate.

Each of the tissue modulating protocols described below is representative of a type of protocol, but is not the only such protocol. In particular, each tissue modulating protocol may, consistent with the type of protocol, be performed with different strain rates and different time values.

As used herein, “stiffness” of a tissue specimen means how much the tissue specimen resists a stretch or strain, which may be measured in pascals, PSI, or similar unit. Thus, “stiffness” of a tissue specimen differs from “stiffness” of a post or other tissue fixture of any of the tissue modulating systems described herein. Furthermore, in a more generalized embodiment of protocols that measure tissue stiffness, a protocol might investigate the stress-strain response of a tissue in response to a strain rate or strain pattern defined by the user, and a proxy of tissue stiffness can be inferred from the stress-strain curve of the tissue.

In some embodiments, one or more tissue modulating protocols induce injury to the tissue specimen, and then subsequent determination of a healing or strengthening protocol is performed, for example with pharmacological treatment. In some embodiments, the tissue modulating protocols characterize the tissue specimens. In some embodiments, the tissue modulating protocols simulate aging or other physiological or pathological changes over time.

In some embodiments, one or more tissue modulating protocols (including the eccentric strain protocols) can be adapted to induce maturation by inducing physiological adaptation (e.g., maturation of an immature muscle or strengthening (aka hypertrophy) of a mature muscle) or pathological adaptation (e.g., injury, fibrosis).

Tissue damage or tissue injury may be established by the detection or measurement of analytes secreted by the tissue specimen, e.g., measurement of cytokines beyond a relevant threshold. Additionally or alternatively, tissue damage or injury may be established by comparing a force exerted by a tissue specimen before and after a tissue modulating protocol. Additionally or alternatively, tissue or injury may be established by sectioning the tissue, dyeing the sections and quantifying the density of healthy muscle fibers, the ratio of cell types or the amount of fibrotic extracellular matrix within the tissue.

Any of method described herein may include an optional step of repeating the method one or more times and optionally controlling, within any relevant time period of the method, any one or more output parameters including tissue stretch (e.g., passive stretch), strain, contraction force, twitch force, muscle fiber recruitment, dwell time, peak displacement, stimulation current, and/or other parameters by varying any one or more input parameters including a stimulation electrode voltage, stimulation current level, stimulation current timing, stimulation input waveform, an input to an actuation assembly of a tissue fixture modulating apparatus, a position and/or movement of one or more tissue fixtures.

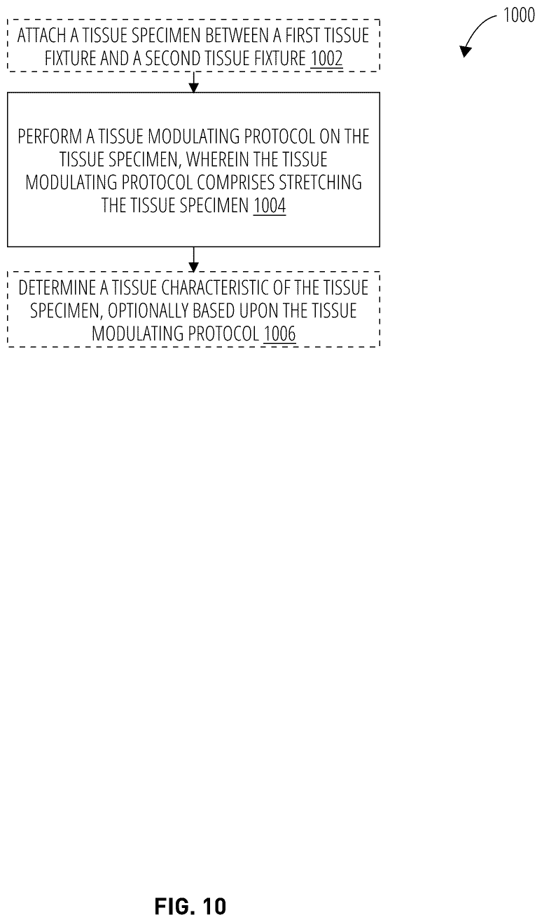

FIG. 10 depicts a method 1000 for modulating a tissue specimen and/or determining a tissue characteristic of a tissue specimen which may be performed independently of the tissue modulating systems described herein or may be performed using any of the tissue modulating systems described herein.

In optional step 1002, method 1000 attaches a tissue specimen between a first tissue fixture (e.g., a rigid post) and a second tissue fixture (e.g., a flexible post).

In step 1004, method 1000 performs a tissue modulating protocol on the tissue specimen, wherein the tissue modulating protocol comprises stretching and/or compressing the tissue specimen, e.g., by actuating the first tissue fixture and the second tissue fixture (such as by separating the first tissue fixture from the second tissue fixture). In some embodiments, the tissue modulating protocol is an eccentric strain protocol, an eccentric stretch during fatigue protocol, an eccentric stretch with partial recruitment protocol, a high strain during relaxation protocol, a repetitive high rate strain no stimulation protocol, an isometric contraction protocol, a stiffness protocol, or a force-length protocol.

In optional step 1006, method 1000 determines a tissue characteristic of the tissue specimen, optionally based upon the tissue modulating protocol performed in step 1004 or a different tissue modulating protocol. In some embodiments, the tissue characteristic is a stiffness, Young's modulus, Poisson's ratio, passive tension and length relationship, contraction force and length relationship, the Frank-Starling relationship, yield strain, failure strain, or similar characteristic or property of the tissue specimen.

In the method 1000 and any method described herein, the determination of a tissue characteristic may be performed by the same or different apparatus or system than performs the tissue modulating protocol. In any embodiment of the method 1000 and any method described herein, the tissue characteristic may be based on the tissue modulating protocol by, for example, basing the determination on a measured deflection of a tissue fixture during or after performance of the protocol. In any embodiment of the method 1000 and any method described herein, the tissue characteristic may be based on the tissue modulating protocol by, for example, determining the tissue characteristic during or after performance of the tissue modulating protocol.

In any method described herein, the optional step of determining a tissue characteristic may be preceded by, may include, or may be replaced by an optional step of measuring a deflection of at least one tissue fixture (e.g., measuring a deflection of a distal end of a flexible post). Thus, any method described herein may conclude with the optional step of measuring a deflection of at least one tissue fixture.

Embodiments include any combination and subcombination of the following optional features.

The first tissue fixture may be a rigid tissue fixture (e.g., rigid post) and the second tissue fixture may be a flexible tissue fixture (e.g., a flexible post).

The tissue modulating protocol may comprise eccentrically stretching the tissue specimen while applying a stimulation current to the tissue specimen. Any method described herein which stretches a tissue specimen while a stimulation current is applied to the tissue specimen eccentrically stretches the tissue specimen. Applying the stimulation current to the tissue specimen may cause the tissue specimen to achieve a fused tetanus state when eccentrically stretched. The stimulation current may cause the tissue specimen to recruit all sarcomeres or fewer than all sarcomeres (partial recruitment) in the fused tetanus state.

Stretching the tissue specimen (eccentrically or otherwise) may be performed by separating the first tissue fixture from the second tissue fixture. Separating the first tissue fixture from the second tissue fixture may be performed by displacing the first tissue fixture (e.g., a rigid post).

The tissue modulating protocol may further comprise allowing the first tissue fixture to move toward the second tissue fixture while applying the stimulation current after separating the first tissue fixture from the second tissue fixture.

Applying the stimulation current to the tissue specimen may cause the tissue specimen to achieve a fused tetanus state, wherein the tissue specimen is in a fatigue state when eccentrically stretched.

The tissue modulating protocol may further comprise allowing the first tissue fixture (e.g., a rigid post or flexible post) to move toward the second tissue fixture (e.g., a flexible post or rigid post) while applying the stimulation current after separating the first tissue fixture from the second tissue fixture.

Stretching the tissue specimen may include stretching the tissue specimen while applying the stimulation current to the tissue specimen, e.g., by repetitively separating the first tissue fixture from the second tissue fixture.

The tissue modulating protocol may comprise applying a stimulation current to the tissue specimen, causing the tissue specimen to achieve a fused tetanus state; and stretching the tissue specimen during a relaxation state when the stimulation current is not applied to the tissue specimen. Stretching the tissue specimen may be performed by separating the first tissue fixture from the second tissue fixture. Separating the first tissue fixture from the second tissue fixture may be performed by displacing a rigid post.

The tissue characteristic may be a stiffness of the tissue specimen, wherein the tissue modulating protocol comprises: applying a strain of a predefined amplitude to the tissue specimen at a steady strain rate by separating the first tissue fixture from the second tissue fixture; and measuring a deflection of the second tissue fixture during and/or after the application of the strain.

The tissue modulating protocol may comprise measuring the tissue characteristic at a plurality of strain values.

The tissue characteristic may be based upon a deflection of at least one of the tissue fixtures.

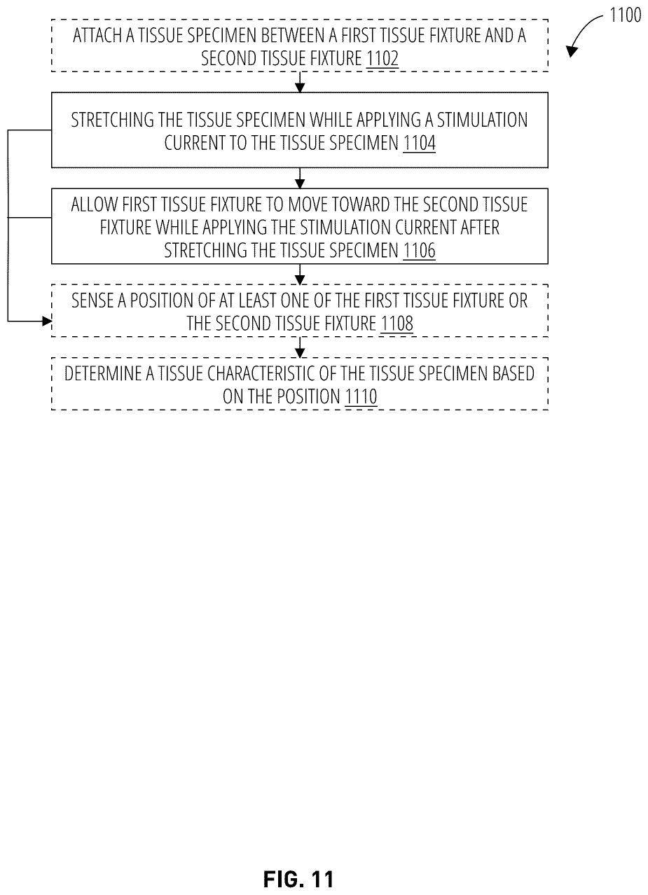

FIG. 11 depicts additional methods for modulating a tissue specimen and/or determining a tissue characteristic of a tissue specimen which may be performed independently of the tissue modulating systems described herein or may be performed using any of the tissue modulating systems described herein.

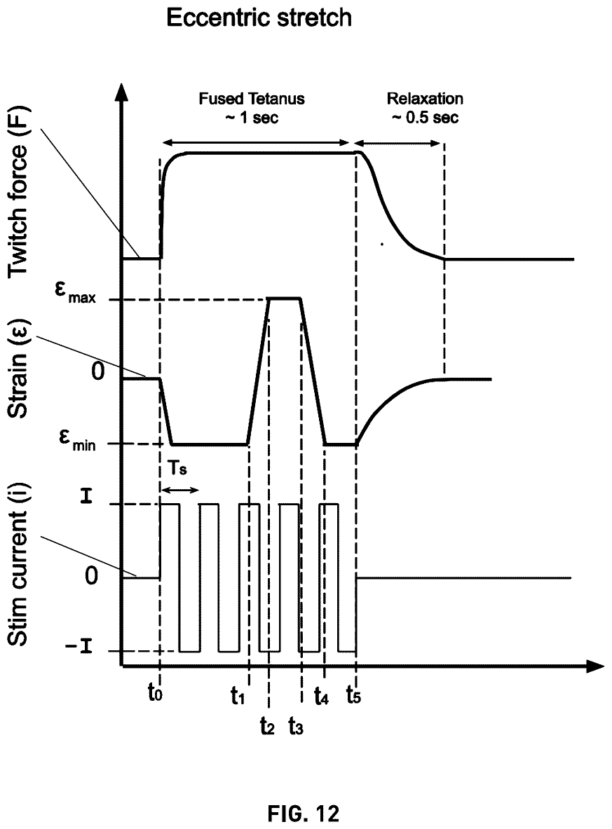

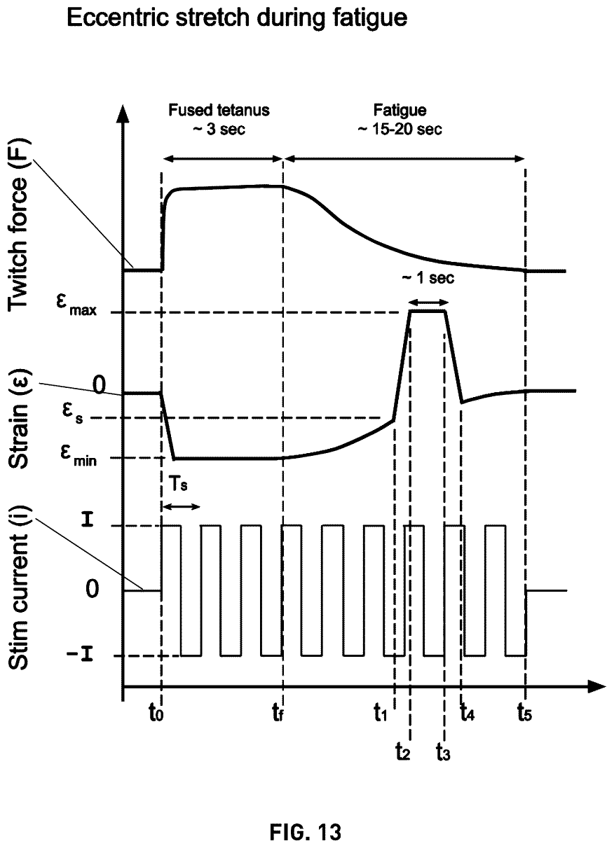

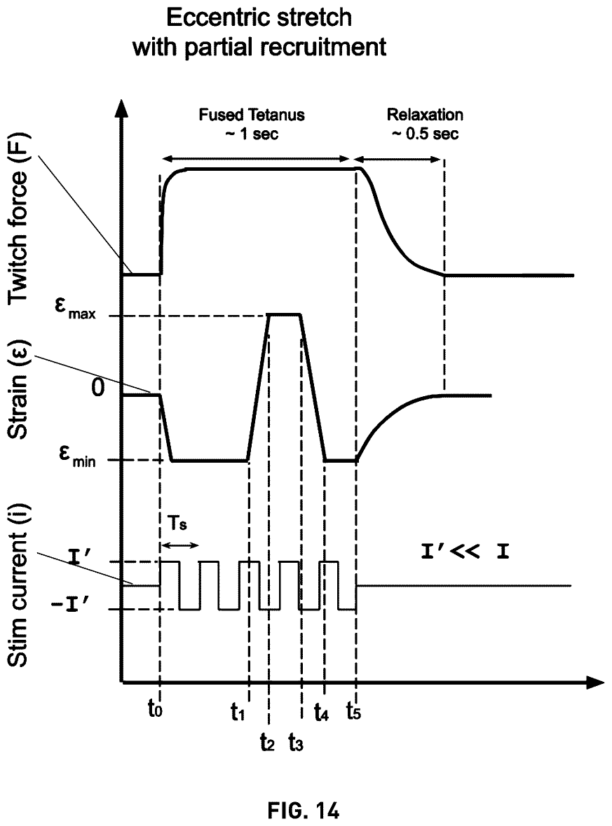

FIG. 11 encompasses different tissue modulating protocols which eccentrically stretch a tissue specimen, including an eccentric strain protocol (see FIG. 12), an eccentric stretch during fatigue protocol (see FIG. 13), and an eccentric stretch with partial recruitment protocol (see FIG. 14).

In optional step 1102, method 1100 attaches a tissue specimen between a first tissue fixture (e.g., a rigid post) and a second tissue (e.g., a flexible post).

In step 1104, method 1100 stretches the tissue specimen while applying a stimulation current to the tissue specimen, e.g., during a fused tetanus state or a fatigue state of the tissue specimen.

In step 1106, method 1100 allows the first tissue fixture (e.g., flexible post) to move toward the second tissue fixture (e.g., rigid post) while applying the stimulation current after stretching the tissue specimen.

In step 1108, method 1100 senses a position of at least one of the first tissue fixture (e.g., flexible post) or the second tissue fixture (e.g., rigid post). Step 1108 may be performed before step 1104, during step 1104, after step 1104, between step 1104 and step 1106, during step 1106, and/or after step 1106.

In optional step 1110, method 1100 determines a tissue characteristic of the tissue specimen, e.g., based on the position sensed in step 1108 and/or based upon a measurement of a property or characteristics of the tissue specimen.

FIG. 12 shows a representative eccentric strain tissue modulating protocol. In some embodiments, the tissue specimen is attached between a first tissue fixture and a second tissue fixture.

At t₀, a stimulation current is applied to the tissue specimen, e.g., utilizing stimulation circuitry of a tissue modulating system. The stimulation current I, frequency 1/T_{S} and pulse length (not shown) should be sufficiently high to induce a fused tetanus. Representative currents include biphasic current or monophasic current.

In a strain phase between t₁ and t₂, while the stimulation current is applied to the tissue specimen, the tissue specimen is stretched until a max strain value is reached at t₂. In some embodiments, the tissue specimen is stretched by actuating the first tissue fixture and/or the second tissue fixture, e.g., by moving the rigid post away from the flexible post.

In some embodiments, the strain change and the strain rate during the upstroke phase [t₁, t₂] of the deflection, are consistent. In some embodiments, a time difference between [t₁, t₂] is on the order of milliseconds, e.g., 100 milliseconds +/−50 milliseconds. The timing t₁ is selected to be repeatable and within the “Fused Tetanus” phase. The stimulation period should be long enough (e.g., at least about 0.5 s) to give ample time for a fused tetanus to develop and to contain the entire upstroke phase, but short enough (e.g., less than about 3 s) to not induce fatigue in the tissue specimen.

In a strain release phase between t₃ and t₄, the strain on the tissue specimen is reduced, e.g., by allowing the first tissue fixture to move toward the second tissue fixture. The strain release phase [t₃, t₄] can precede, include or follow the end of stimulation t₅.

To mitigate elevated tissue stiffness during the “fused tetanus” phase, a tissue fixture modulating apparatus performing the foregoing tissue modulating protocol may optionally include a tissue fixture limiter for temporarily fixturing one tissue fixture (e.g., flexible post) to increase the stiffness of the tissue fixture to facilitate stretching of the tissue specimen.

FIG. 13 shows another eccentric strain tissue modulating protocol, i.e., an eccentric stretch during fatigue protocol for a tissue specimen. In some embodiments, the tissue specimen is attached between a first tissue fixture and a second tissue fixture.

This tissue modulating protocol overcomes the high tissue specimen stiffness by prolonging the electrical stimulation and postponing eccentric strain until the tissue specimen enters a fatigue state, when tissue stiffness may decrease. The electrical stimulation is configured to achieve fused tetanus and is performed over a time period between t₀ and t₅, (e.g., about 20 seconds).

At t₀, a stimulation current is applied to the tissue specimen, e.g., utilizing stimulation circuitry of a tissue modulating system. The stimulation current I, frequency 1/T_{S} and pulse length (not shown) should be sufficiently high to induce a fused tetanus.

At t_{f}, the tissue specimen begins to fatigue as evidenced by reduced strain.

In a strain phase between t₁ and t₂, while the stimulation current is applied to the tissue specimen and the tissue specimen is in the fatigue state, the tissue specimen is stretched until a max strain value is reached at t₂. In some embodiments, the tissue specimen is stretched by actuating the first tissue fixture and/or the second tissue fixture, e.g., by moving the rigid post away from the flexible post.

In this tissue modulating protocol, t₁ should be selected to be within the fatigue state, preferably early therein (e.g., at a specific distance from the onset of fatigue t_{f} or at the moment of a predefined percent reduction of the twitch force (or % reduction of shortening).

Parameters of the tissue modulating protocol may be varied, including the strain change and the strain rate during the upstroke phase [t₁, t₂] of the deflection (it may be the same as in FIG. 12) and the timing between t_{f} and t₁.

FIG. 14 shows still another representative eccentric strain tissue modulating protocol, i.e., an eccentric stretch with partial recruitment protocol. “Partial recruitment” refers to the tissue specimen utilizing less than all of its sarcomeres of the tissue specimen. In some embodiments, the tissue specimen is attached between a first tissue fixture and a second tissue fixture. This protocol is similar to the protocol of FIG. 12 and mitigates high tissue specimen stiffness by reducing the stimulation current amplitude I′ (or pulse length) to enable partial recruitment of sarcomeres and still attain a fused tetanus state.

The electrical stimulation is applied long enough for the fused tetanus to develop. Relevant parameters include the strain change and the strain rate during the upstroke phase [t₂, t₃] of the deflection (it may be the same as in FIG. 12) and the timing between t₁ and t₂. In this tissue modulating protocol, t₂ is selected such that the upstroke phase is contained within the fused tetanus phase.

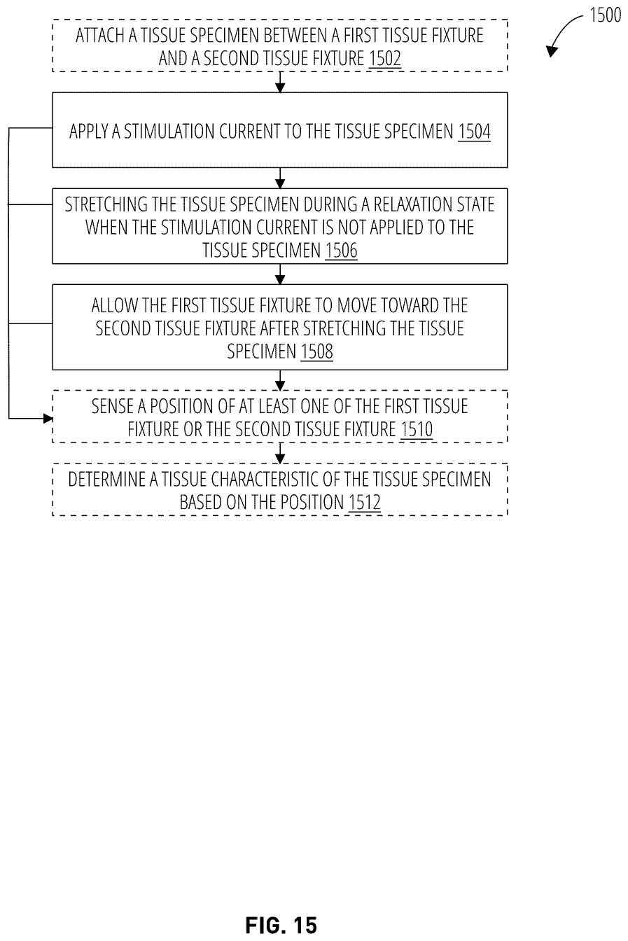

FIG. 15 depicts additional methods for modulating a tissue specimen and/or determining a tissue characteristic of a tissue specimen which may be performed independently of the tissue modulating systems described herein or may be performed using any of the tissue modulating systems described herein.

FIG. 15 encompasses different tissue modulating protocols which stretch a tissue specimen when a stimulation current is not applied, e.g., a high strain during relaxation protocol.

In optional step 1502, method 1500 attaches a tissue specimen between a first tissue fixture (e.g., rigid post) and a second tissue (e.g., flexible post).

In step 1504, method 1500 applies a stimulation current to the tissue specimen, e.g., causing the tissue specimen to achieve a fused tetanus state.

In step 1506, method 1500 stretches the tissue specimen during a relaxation state when the stimulation current is not applied to the tissue specimen.

In step 1508, method 1500 allows the first tissue fixture (e.g., rigid post) to move toward the second tissue fixture (e.g., flexible post) after stretching the tissue specimen.

In optional step 1510, method 1500 senses a position of at least one of the first tissue fixture or the second tissue. Step 1510 may be performed before step 1504, during step 1504, after step 1504, between step 1504 and step 1506, during step 1506, after step 1506, between step 1506 and step 1508, during step 1508, and/or after step 1508.

In optional step 1512, method 1500 determines a tissue characteristic of the tissue specimen, e.g., based on the position sensed in step 1510 and/or based upon a measurement of a property or characteristics of the tissue specimen.

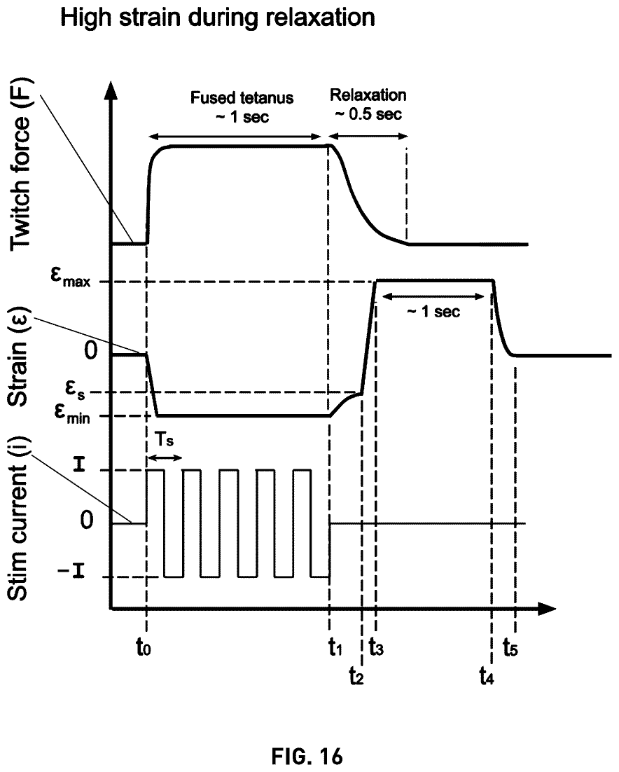

FIG. 16 shows another representative tissue modulating protocol for a tissue specimen, i.e., a high strain during relaxation protocol. Such protocol may be an advantageous alternative to the partial recruitment protocol shown in FIG. 14 for certain tissue specimens which do not respond well to the partial recruitment protocol. Restated, the protocol of FIG. 16 enables time-control over partial recruitment, rather than electrical current-control over partial recruitment. The protocol combines the above description with the approach of the “Partial recruitment” protocol to further reduce the stiffness during the relaxation phase. This protocol mitigates high tissue specimen stiffness by postponing eccentric strain until after the end of stimulation, where the tissue specimen is in the process of relaxing. Tissue stiffness will gradually decrease during the relaxation phase.

Between t₀ and t₁, a stimulation current is applied to the tissue specimen, e.g., utilizing stimulation circuitry of a tissue modulating system. The stimulation current I, frequency 1/T_{S} and pulse length (not shown) should be sufficiently high to induce a fused tetanus (either complete or partial recruitment).

At t₁, the stimulation current is stopped and the tissue specimen enters a relaxation state.

At t₂, the tissue specimen is stretched until a max strain value is reached at t₃. In some embodiments, the tissue specimen is stretched by actuating the first tissue fixture and/or the second tissue fixture, e.g., by moving the rigid post away from the flexible post.

Relevant parameters include the strain change and the strain rate during the upstroke phase [t₂, t₃] of the deflection (it may be the same as in FIG. 12) and the timing between t₁ and t₂. In this protocol, t₂ should be within the relaxation phase. Given the short duration of the relaxation phase, automated synchronization of stimulation and strain may be utilized so t₂-t₁ has a reproducible precision of ˜100 ms.

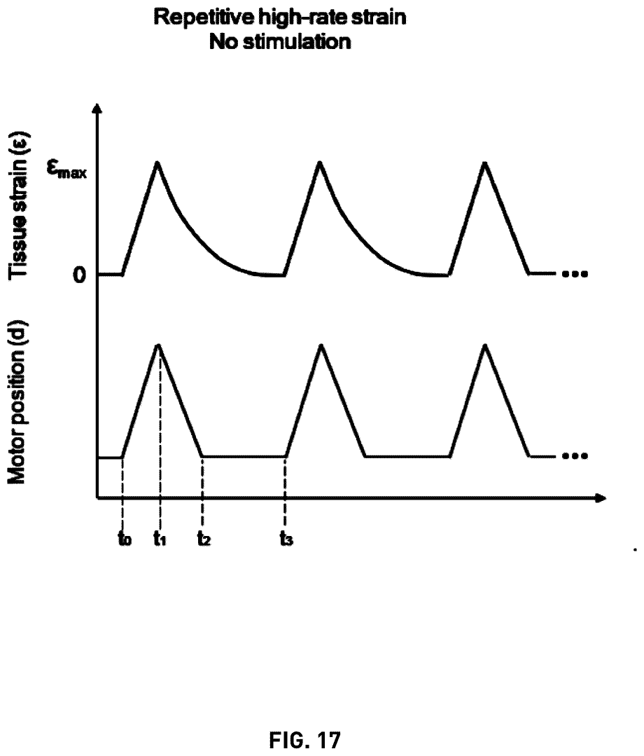

FIG. 17 shows a repetitive high rate strain no stimulation tissue modulating protocol.

This tissue modulating protocol mitigates high tissue specimen stiffness by not electrically stimulating the tissue specimen altogether. The load is applied at resting state (without stimulation), and a fraction of motor units that are spontaneously in the bound state will experience the strain. Since this is a fraction of the strained motor units of the Ideal eccentric strain load, the strain may be applied significantly more times to cause the same level of injury to the entire muscle.

The strain is applied repeatedly at a high frequency. A triangular pulse of strain is applied (no steady plateau phase) to minimize the amount of idle time in a pulse. In some embodiments, the strain amplitude and stain rate are the same as in the FIG. 12 protocol. In some embodiments, the resting state phase [t₂, t₃] is selected so that the tissue fully relaxes before the onset of the next strain pulse at t₃.

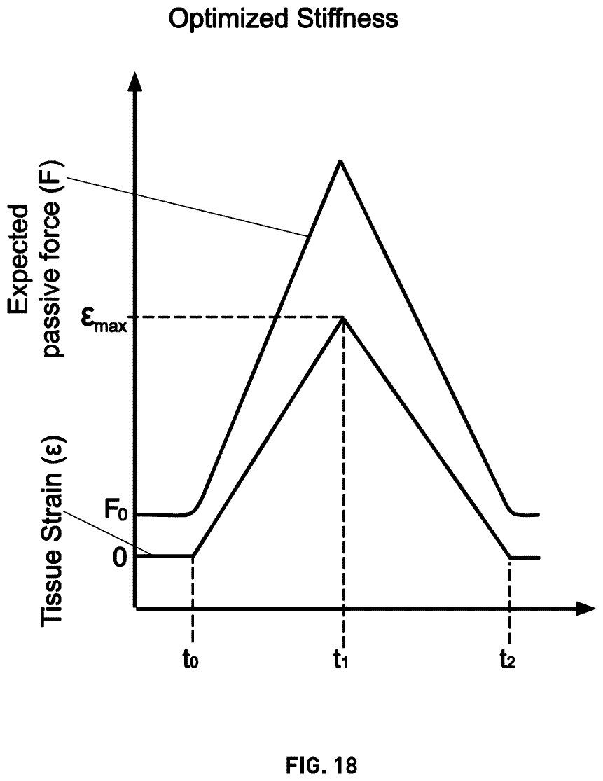

FIG. 18 shows a stiffness tissue modulating protocol. This tissue modulating protocol measures the passive stiffness of a tissue by applying a low amplitude strain at a steady strain rate and measuring the resulting post deflection. The post deflection is converted to the force passively exerted on the post by the elongated tissue, and the tissue stiffness is subsequently calculated as the slope of the passive force.

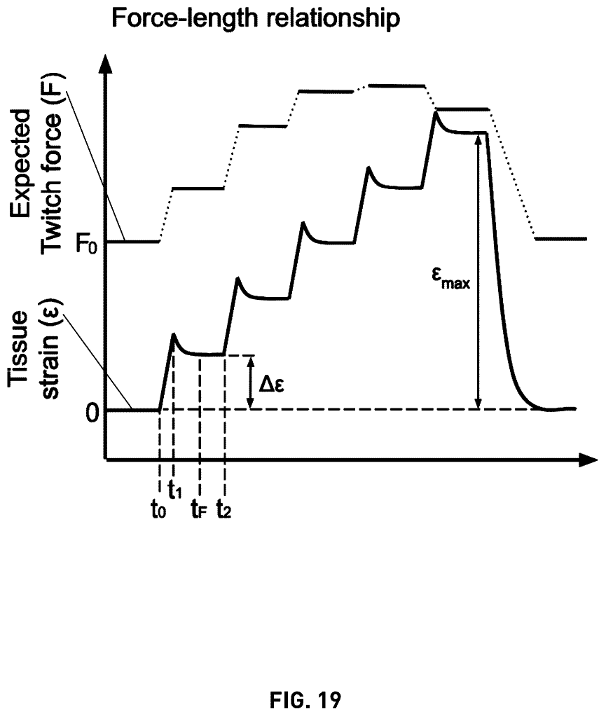

FIG. 19 shows a force-length tissue modulating protocol. This protocol quantifies the force-length relationship of a tissue specimen by gradually increasing the strain of the tissue and measuring the tissue twitch force at each strain value. The force can be measured anytime within [t_{f}, t₂], during which the transient behavior of the tissue fixture/tissue specimen (seen within [t₁, tF]) has ended and the strain can be considered constant.

Thus far, the present disclosure has described tissue modulating systems and tissue fixture modulating apparatuses as tools that stretch tissue specimens. However, tissue fixture modulating apparatuses, systems, and tissue modulating protocols of the present disclosure can alternatively compress tissue specimens. In other words, tissue modulating systems and tissue fixture modulating apparatuses of the present disclosure can modulate a tissue length in either direction.

For example, a tissue fixture modulating apparatus can include a mechanism that changes the resting state of the first tissue fixture such as a rigid post and/or flexible post (e.g., via a spring-loaded piston). The tissue fixture modulating apparatus may include a mechanism to control the motion limits of the displaced tissue fixture, allowing for a tighter or broader range of tissue fixture position control by moving either the position of stretch initiation or the position of the max stretch. Such mechanisms include, for example, tissue fixtures including the tissue fixture limiter 330 of FIG. 3.

In a system as described above in which a tissue specimen is suspended between two tissue fixtures (e.g., between a rigid post and a flexible post), the flexible post may be bent because the tissue (even though it is actively not contracting) has some internal tension. The length of the resting tissue is achieved when the internal tension of the tissue matches the bending resistance of the flexible post. In this state, the tissue specimen is not dynamically stretched. In this state, the cells in the tissue experience a passive tension and have a particular length. However, the tissue specimen may reach a shorter state.

Moving the two tissue fixtures closer together produces two results. In that moment, one or both tissue fixtures (e.g., the flexible post) will not bend as much, so the tissue specimen will experience less tension from the flexible post. The tissue specimen might temporarily lose its tension at that time, but over time (e.g., hours/days), the tissue specimen will try to compact itself, bending the flexible post again. At some point, a new equilibrium will be reached, where the internal tension of the tissue (could be a different value than before) will match the bending resistance of the flexible post. This equilibrium is a new resting state of the tissue. The two resting states are physiologically different and lead to different tissue properties. Therefore, tissue modulating systems of the present disclosure may be configured to measure the new properties and the relevant changes.

Briefly after moving the post, the resistance a contracting tissue specimen will meet while contracting (afterload) is likely to be lower than before. The tension it experiences at the onset of contraction (preload) is lower too. The cells in the tissue might not be as stretched when they decide to contract (again, preload). The resulting contraction will be different compared to a contraction from the original resting state. Therefore, tissue modulating systems of the present disclosure may be configured to measure the new contraction and the relevant changes.

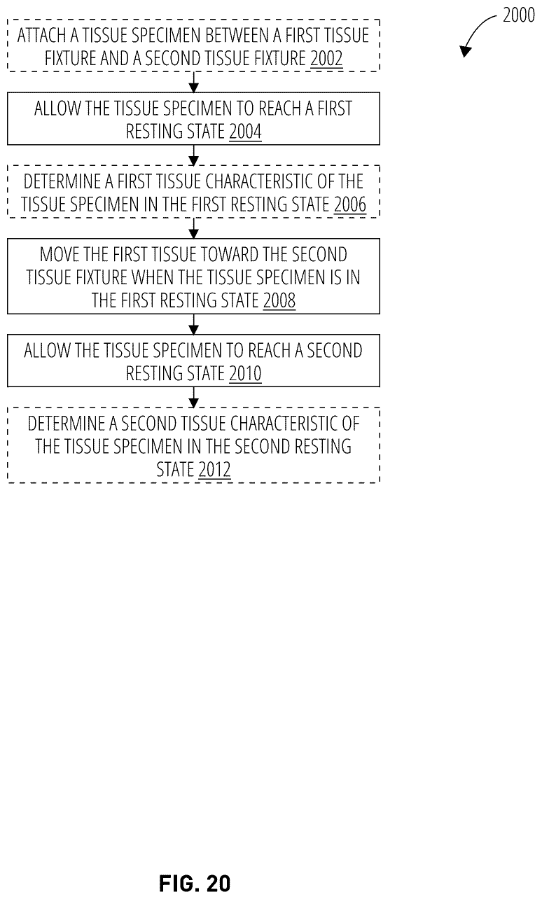

FIG. 20 depicts additional methods for modulating a tissue specimen and/or determining a tissue characteristic of a tissue specimen which may be performed independently of the tissue modulating systems described herein or may be performed using any of the tissue modulating systems described herein.

In optional step 2002, method 2000 attaches a tissue specimen between a first tissue fixture and a second tissue fixture. In step 2004, method 2000 allows the tissue specimen to reach a first resting state. In optional step 2006, method 2000 determines a first tissue characteristic of the tissue specimen in the first resting state. In step 2008, method 2000 moves the first tissue toward the second tissue fixture when the tissue specimen is in the first resting state. In step 2010, method 2000 allows the tissue specimen to reach a second resting state. In optional step 2012, method 2000 determines a second tissue characteristic of the tissue specimen in the second resting state.

Any tissue modulating protocol may be further enhanced through feedback, e.g., utilizing a feedback system of the tissue modulating system. For example, any method described herein may be sequentially repeatedly based upon feedback that modulates parameters such as the stimulation current, strain rate, and time periods, e.g., to prevent eccentric stretching to avoid unwanted tissue injury.

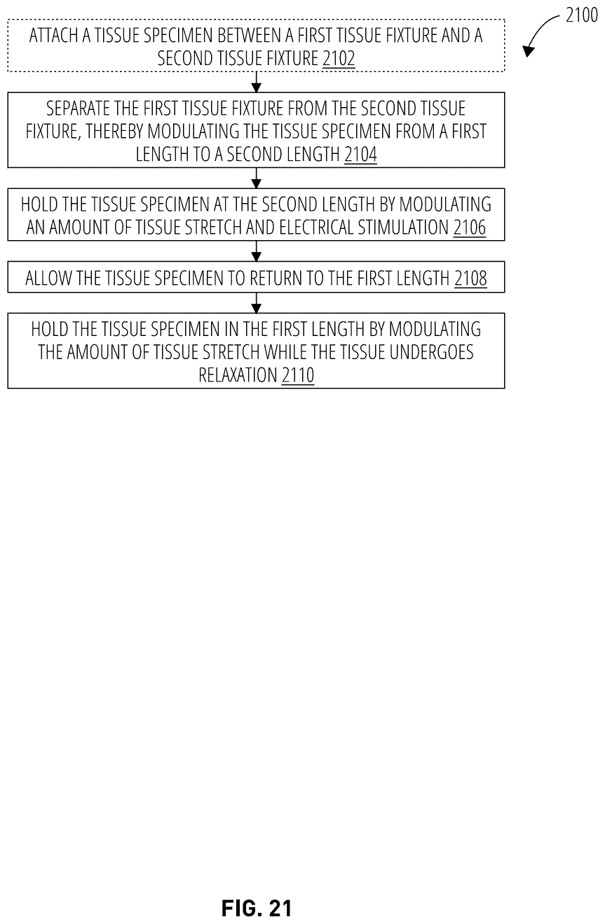

FIG. 21 depicts additional methods for modulating a tissue specimen and/or determining a tissue characteristic of a tissue specimen which may be performed independently of the tissue modulating systems described herein or may be performed using any of the tissue modulating systems described herein.

FIG. 21 illustrates a tissue modulating protocol that simulates a pressure-volume relationship of a cardiac tissue model wherein the timing of a tissue stretch is controlled via real-time feedback. Such a simulation may be performed by stretching one or more tissue specimens utilizing any tissue modulating system described herein, where tissue stretch is a proxy for atrial or ventricular volume and wherein tissue force is a proxy for atrial and ventricular pressure. Such simulation can effectively condition tissue specimens to simulate cardiac tissue.

For example, a closed-loop tissue modulating system of this disclosure implemented for such application provides real-time feedback to the actuators of the apparatus to coordinate the stretching of one or more tissue specimens to take place during a relaxation stage of contraction, based on the measurements of displacement and other contraction characteristics of the beating cardiac tissues. The closed-loop input can be tuned to provide control or other contraction protocols such that the stretch would not take place until a “maximum contraction” occurs to minimize potential damage or injury to the contracting tissues due to the excessive stretch. Then, as the cardiac tissue enters a relaxation stage, a stretch control waveform is applied to cause the tissue to quickly reach the peak stretch displacement and then return to the baseline (initial position or distance under passive tension) at a rate equal to or slightly faster than the speed of tissue contraction.

Such a closed-loop system can be implemented to modulate a tissue specimen, e.g., by providing an engineered cardiac system wherein the apparatus of this disclosure is used to simulate isovolumetric contraction and relaxation that heart tissue undergoes during a cardiac cycle.

FIG. 21 illustrates a representative method of determining a tissue characteristic or modulating a tissue specimen, as described above.

In optional step 2102, method 2100 attaches a tissue specimen between a first tissue fixture (e.g., a rigid post) and a second tissue fixture (e.g., a flexible post).

In step 2104, method 2100 separates the first tissue fixture from the second tissue fixture, thereby modulating the tissue specimen from a first length to a second length. Step 2104 may simulate a diastolic filling phase, e.g., by stretching the tissue from a less extended state to a more extended state.

In step 2106, method 2100 holds the tissue specimen at the second length by modulating an amount of tissue stretch and electrical stimulation. Step 2106 may simulate an isovolumetric contraction phase, e.g., by holding the tissue specimen in the more-extended state and continuously adjusting the amount of tissue stretch via the closed loop control to pull the tissue to maintain the same extended state while the tissue undergoes contraction.

In step 2108, method 2100 allows the tissue specimen to return to the first length. Step 2108 may simulate a ventricular ejection phase, e.g., by allowing the tissue to contract and return to the less extended state and, optionally, adjusting the amount of tissue stretch to apply varying afterload forces to the tissue while it contracts.

In step 2110, method 2100 holds the tissue specimen in the first length by modulating the amount of tissue stretch while the tissue undergoes relaxation. Step 2110 may simulate an isovolumetric relaxation phase, e.g., by holding the tissue in the less extended state and continuously adjusting the amount of tissue stretch via the closed loop control to push the tissue to maintain the same less extended state while the tissue undergoes relaxation.

Similarly, the closed loop feedback systems can also be used to coordinate temporal stimulation of the tissues (e.g., by way of electrical stimulation using the electrodes disclosed herein) with stretching to produce or replicate complex protocols for tissue conditioning. One example is a tissue modulating protocol that induces or simulates eccentric contraction such that a stimulation-induced contraction occurs and overlaps with a mechanically induced stretching of the tissues.

Input parameters including stretch and stimulation with various amplitudes, waveforms, and/or synchronicities can be combined in many different combinations to produce different tissue phenotypes in different wells of a multi-well plate (e.g., 24-or 96-well plates). Moreover, the resulting tissue displacements that take place during stretching and contraction phases can be measured and used as feedback to provide real-time control over the conditioning protocol and accordingly adjust to the changing mechanical or phenotypical properties of the tissues induced by said tissue modulating protocols.

Based on the foregoing description of the disclosure, the systems and apparatuses of this disclosure can also be used to develop stretch or conditioning protocols for producing more physiologically mature contractile tissues. For example, the apparatuses of this disclosure can be used to stretch tissue specimens for N cycles according to an input stretch profile and then to suspend stretching for a period of time during which contractile forces and other contraction characteristics of the tissues are measured, via the feedback systems described above. Based on real-time feedback from such measurements, the input stretch profile can be modified to further evaluate the effects of varying stretch protocols on the physiological maturation of the tissues or to develop optimal stretch protocols that can produce more physiologically mature tissues having greater contractile forces.

Such feedback-based “intelligent” maturation or conditioning strategies can be further enhanced with machine-learning algorithms which are part of or executed in connection with tissue modulating systems of the present disclosure. For example, machine learning can be applied to train the tissue modulating systems to measure the tissue's contractile forces, maturation markers, and other tissue characteristics upon application of different stretch or conditioning protocols, for example, by applying different independent variables for stretch control such as dwell time, peak displacement, asymmetry of sinusoidal input waveform, etc., to optimize or predict the results of optimization for a dependent variable such as the time to condition the tissue to maturation, or contractile strength of the tissue. Representative tissue modulating protocols are provided below. For example, such machine learning algorithms may determine the best tissue modulating protocol to induce maturation of tissues.

With the use of such machine learning techniques, together with the imaging analysis algorithms and the feedback-based control for mechanical conditioning of engineered 3D tissues, this disclosure provides an innovative strategy for developing or producing more mature, physiologically relevant, human 3D tissue models in a highly efficient manner. The tissue modulating systems of this disclosure thus provide an essential in vitro tool for evaluating the effects of various therapeutic strategies or treatments, particularly, for the diseases involving disorders affecting cardiac, skeletomuscular, neuromuscular, and other muscular functions of the human body.

It should be noted that various changes can be made to the embodiments of this disclosure as could be reasonably contemplated, in view of the above-described description, by any person skilled in the art. The following claims are presented as examples of embodiments of this disclosure, but these claims should not be construed to limit other claims or other embodiments disclosed herein.

The present application may reference quantities and numbers. Unless specifically stated, such quantities and numbers are not to be considered restrictive, but representative of the possible quantities or numbers associated with the present application. Also, in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrases “at least one of A, B, and C,” “at least one of A, B, or C,” or similar expressions referencing two or more elements includes: (A), (B), (C), (A and B), (A and C), (B and C), or (A, B, and C), including all further possible permutations when greater than three elements are listed.

Embodiments disclosed herein may utilize circuitry in order to implement technologies and methodologies described herein, operatively connect two or more components, generate information, determine operation conditions, control an appliance, device, or method, and/or the like. Circuitry of any type can be used. In an embodiment, circuitry includes, among other things, one or more computing devices such as a processor (e.g., a microprocessor), a central processing unit (CPU), a digital signal processor (DSP), an application-specific integrated circuit (ASIC), a field-programmable gate array (FPGA), or the like, or any combinations thereof, and can include discrete digital or analog circuit elements or electronics, or combinations thereof.

In an embodiment, circuitry includes one or more ASICs having a plurality of predefined logic components. In an embodiment, circuitry includes one or more FPGA having a plurality of programmable logic components. In an embodiment, circuitry includes hardware circuit implementations (e.g., implementations in analog circuitry, implementations in digital circuitry, and the like, and combinations thereof). In an embodiment, circuitry includes combinations of circuits and computer program products having software or firmware instructions stored on one or more computer readable memories that work together to cause a device to perform one or more methodologies or technologies described herein. In an embodiment, circuitry includes circuits, such as, for example, microprocessors or portions of microprocessor, that require software, firmware, and the like for operation. In an embodiment, circuitry includes an implementation comprising one or more processors or portions thereof and accompanying software, firmware, hardware, and the like. In an embodiment, circuitry includes a baseband integrated circuit or applications processor integrated circuit or a similar integrated circuit in a server, a cellular network device, other network device, or other computing device. In an embodiment, circuitry includes one or more remotely located components. In an embodiment, remotely located components are operatively connected via wireless communication. In an embodiment, remotely located components are operatively connected via one or more receivers, transmitters, transceivers, or the like.

An embodiment includes one or more data stores that, for example, store instructions or data. Non-limiting examples of one or more data stores include volatile memory (e.g., Random Access memory (RAM), Dynamic Random Access memory (DRAM), or the like), non-volatile memory (e.g., Read-Only memory (ROM), Electrically Erasable Programmable Read-Only memory (EEPROM), Compact Disc Read-Only memory (CD-ROM), or the like), persistent memory, or the like. Further non-limiting examples of one or more data stores include Erasable Programmable Read-Only memory (EPROM), flash memory, or the like. The one or more data stores can be connected to, for example, one or more computing devices by one or more instructions, data, or power buses.

In an embodiment, circuitry includes one or more computer-readable media drives, interface sockets, Universal Serial Bus (USB) ports, memory card slots, or the like, and one or more input/output components such as, for example, a graphical user interface, a display, a keyboard, a keypad, a trackball, a joystick, a touch-screen, a mouse, a switch, a dial, or the like, and any other peripheral device. In an embodiment, circuitry includes one or more user input/output components that are operatively connected to at least one computing device to control (electrical, electromechanical, software-implemented, firmware-implemented, or other control, or combinations thereof) one or more aspects of the embodiment.

In an embodiment, circuitry includes a computer-readable media drive or memory slot configured to accept signal-bearing medium (e.g., computer-readable memory media, computer-readable recording media, or the like). In an embodiment, a program for causing a system to execute any of the disclosed methods can be stored on, for example, a computer-readable recording medium (CRMM), a signal-bearing medium, or the like. Non-limiting examples of signal-bearing media include a recordable type medium such as any form of flash memory, magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., transmitter, receiver, transceiver, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

The detailed description set forth above in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result. Generally, the embodiments disclosed herein are non-limiting, and the inventors contemplate that other embodiments within the scope of this disclosure may include structures and functionalities from more than one specific embodiment shown in the figures and described in the specification.

In the foregoing description, specific details are set forth to provide a thorough understanding of exemplary embodiments of the present disclosure. It will be apparent to one skilled in the art, however, that the embodiments disclosed herein may be practiced without embodying all the specific details. In some instances, well-known process steps have not been described in detail in order not to unnecessarily obscure various aspects of the present disclosure. Further, it will be appreciated that embodiments of the present disclosure may employ any combination of features described herein.

The present application may include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive, it will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

1. A tissue modulating system configured for use with a multi-well plate comprising a plurality of wells, the tissue modulating system comprising:
a first plurality of tissue fixtures, wherein at least one tissue fixture of the first plurality of tissue fixtures is configured to extend into each well of the plurality of wells; and
a tissue fixture modulating apparatus comprising a second plurality of tissue fixtures, wherein at least one tissue fixture of the second plurality of tissue fixtures configured to extend into each well of the plurality of wells, wherein the tissue fixture modulating apparatus is configured to displace, within each well, the at least one tissue fixture of the second plurality of tissue fixtures.

2. The tissue modulating system of claim 1, wherein each tissue fixture of the second plurality of tissue fixtures has a greater stiffness than each tissue fixture of the first plurality of tissue fixtures.

3. The tissue modulating system of claim 2,
wherein the first plurality of tissue fixtures is a plurality of flexible posts,
wherein the second plurality of tissue fixtures is a plurality of rigid posts.

4. The tissue modulating system of claim 1, wherein each tissue fixture of the first plurality of tissue fixtures has a greater stiffness than each tissue fixture of the second plurality of tissue fixtures.

5. The tissue modulating system of claim 4,
wherein the first plurality of tissue fixtures is a plurality of rigid posts,
wherein the second plurality of tissue fixtures is a plurality of flexible posts.

6. The tissue modulating system of claim 2-or claim 4, further comprising a well plate lid, wherein at least one of the first plurality of tissue fixtures or the second plurality of tissue fixtures attaches to the well plate lid.

7. The tissue modulating system of claim 6, wherein at least one of the first plurality of tissue fixtures or the second plurality of tissue fixtures extends through the well plate lid.

8. The tissue modulating system of claim 2-or claim 4, wherein the second plurality of tissue fixtures pivots about a pivot point.

9. The tissue modulating system of claim 8, wherein the second plurality of tissue fixtures extends away from a beam coupled with the pivot point.

10. The tissue modulating system of claim 9, wherein the tissue fixture modulating apparatus comprises an actuator configured to pivot or translate the beam.

11. The tissue modulating system of claim 10, wherein the actuator contacts a lever extending away from the beam.

12. The tissue modulating system of claim 8, further comprising a plurality of tissue fixture limiters configured to extend into the plurality of wells.

13. The tissue modulating system of claim 12, wherein a distal end of each tissue fixture limiter is configured to selectively intercept movement of at least one of the second plurality of tissue fixtures.

14. The tissue modulating system of claim 12, wherein the plurality of tissue fixture limiters is independently movable between a fully retracted position and a fully deployed position.

15. The tissue modulating system of claim 12, further comprising the plurality of tissue fixture limiters.

16. The tissue modulating system of claim 8, further comprising a plurality of electrodes configured to extend into the plurality of wells.

17. The tissue modulating system of claim 2-or claim 4, wherein the tissue modulating apparatus comprises an actuator configured to displace the second plurality of tissue fixtures.

18. The tissue modulating system of claim 2-or claim 4, wherein the tissue modulating apparatus comprises a plurality of actuators, each actuator of the plurality of actuators being configured to displace a different tissue fixture of the second plurality of tissue fixtures.

19. The tissue modulating system of claim 18, further comprising a non-transitory computer-readable storage medium storing instructions that when executed by a processor cause the processor to actuate each actuator independently from each other actuator of the plurality of actuators.

20. The tissue modulating system of claim 1, further comprising a non-transitory computer-readable storage medium storing instructions that when executed by a processor cause the processor to actuate the tissue fixture modulating apparatus.

21-44. (canceled)
